(19) United States
(12) Patent Application Publication
(10) Pub. No.: US 2023/0248241 A1
(43) Pub. Date: Aug. 10, 2023

(54) Medical Systems, Devices, and Kits Useful in Performing Treatment under Magnetic Resonance Imaging and Related Methods

(71) Applicants: Eric D. Anttila, West Lafayette, IN (US); Grant M. Baker, Lafayette, IN (US); Eric Brandner, West Lafayette, IN (US); Sean D. Chambers, Bloomington, IN (US); Neal Fearnot, West Lafayette, IN (US); David C. Gross, Lafayette, IN (US); Joshua F. Krieger, Topsfield, MA (US); Ram H. Paul, JR., Bloomington, IN (US); Andrew J. Robison, Otterbein, IN (US); Jesse Roll, West Lafayette, IN (US)

(72) Inventors: Eric D. Anttila, West Lafayette, IN (US); Grant M. Baker, Lafayette, IN (US); Eric Brandner, West Lafayette, IN (US); Sean D. Chambers, Bloomington, IN (US); Neal Fearnot, West Lafayette, IN (US); David C. Gross, Lafayette, IN (US); Joshua F. Krieger, Topsfield, MA (US); Ram H. Paul, JR., Bloomington, IN (US); Andrew J. Robison, Otterbein, IN (US); Jesse Roll, West Lafayette, IN (US)

(21) Appl. No.: 18/106,601

(22) Filed: Feb. 7, 2023

Related U.S. Application Data
(60) Provisional application No. 63/307,369, filed on Feb. 7, 2022.

(51) Int. Cl. A61B 5/00; A61B 90/00; A61B 5/055; A61B 10/02

(52) U.S. Cl. CPC A61B 5/0036; A61B 5/055; A61B 10/0233; A61B 90/39; A61B 2090/3954

(57) ABSTRACT

Example medical systems for performing treatment under magnetic resonance imaging and related devices, kits, and methods are described. An example medical system includes a first medical device, a second medical device, and a marker. The first medical device is formed of a first material. The first material has a first magnetic susceptibility. The second medical device is formed of a second material. The second material has a second magnetic susceptibility. The marker is disposed on the second medical device and is formed of a third material. The third material has a third magnetic susceptibility that is different than the first magnetic susceptibility and the second magnetic susceptibility.

### FIELD

The disclosure relates generally to the field of medical systems and devices. More particularly, the disclosure relates to medical systems useful in performing treatment under magnetic resonance imaging (MRI), medical devices useful in performing treatment under MRI, kits useful in performing treatment under MRI, and methods of performing interventional medical treatment under MRI.

### BACKGROUND

The field of interventional MRI is gaining wider acceptance and seeing an increase in the number of procedures that can be performed. Interventional procedures conducted under MRI have several benefits over X-Ray-guided interventions. For example, the patient is not exposed to ionizing radiation. Also, MRI provides the ability to characterize tissue and functional flow during an interventional procedure.

The development of interventional procedures conducted under MRI has been limited as a result of the tools needed to perform these procedures being unavailable. Therefore, patients are required to make multiple visits to treatment facilities to visualize, diagnose, and treat various conditions. In addition, multiple imaging modalities are often needed, which impacts the accuracy of utilizing a magnetic resonance (MR) image in directing intervention. For example, when addressing prostate cancer, visualization, biopsy, and treatment are currently completed over the course of three patient visits. At a first visit, a scan is completed using a MR scanner to produce an image showing the prostate and any abnormalities. The patient then leaves the facility and awaits a review of the image. If abnormalities exist, a second patient visit will occur such that a biopsy sample of the abnormal tissue can be completed. Currently, software is used to fuse the MR image with the procedural ultrasound to provide guidance in conducting the biopsy. This fusion decreases the value of the diagnostic MR image. The patient then leaves the facility again and awaits a review of the biopsy sample to determine whether further treatment is required (e.g., if the review results in a positive prostate cancer diagnosis). If further treatment is required, the patient will visit the facility a third time such that treatment can be performed. Completion of these three patient visits can take months, prevents the patient from receiving rapid treatment, and increases the overall costs associated with treatment. Furthermore, software used to fuse MR images with other images (e.g., those obtained via ultrasound) have drawbacks, such as potential image overlay issues and the potential for compression shifting of tissues (e.g., prostate).

A need exists, therefore, for new and improved medical systems useful in performing treatment under MRI, medical devices useful in performing treatment under MRI, kits useful in performing treatment under MRI, and methods of performing interventional medical treatment under MRI.

### SUMMARY OF SELECTED EXAMPLE EMBODIMENTS

Various example medical systems useful in performing treatment under MRI, medical devices useful in performing treatment under MRI, kits useful in performing treatment under MRI, and methods of performing interventional medical treatment under MRI are described herein.

An example medical system useful in performing treatment under MRI includes a first medical device, a second medical device, and a marker. The first medical device is formed of a first material. The first material has a first magnetic susceptibility. The second medical device is formed of a second material. The second material has a second magnetic susceptibility. The marker is disposed on the second medical device and is formed of a third material. The third material has a third magnetic susceptibility that is different than the first magnetic susceptibility and the second magnetic susceptibility.

An example medical device useful in performing treatment under MRI includes an elongate member and a plug. The elongate member has a proximal end, a distal end, and a main body formed of a first material that has a first magnetic susceptibility. The plug is disposed on the elongate member and comprises a second material disposed within a third material. The second material has a second magnetic susceptibility. The third material has a third magnetic susceptibility. The second magnetic susceptibility is different than the first magnetic susceptibility and the third magnetic susceptibility.

An example kit useful in performing treatment under magnetic resonance includes a first medical device, a second medical device, and a third medical device. The first medical device includes a first marker that produces a first image artifact under a first MR field strength. The second medical device includes a second marker that produces a second image artifact under a second MR field strength. The third medical device includes a third marker that produces a third image artifact under a third MR field strength. The first MR field strength is different than the second MR field strength and the third MR field strength. The second MR field strength is different than the third MR field strength. The first image artifact has a first set of characteristics (e.g., size, shape, pattern, and/or intensity) under the first MR field strength, a second set of characteristics under the second MR field strength, and a third set of characteristics under the third MR field strength. The first set of characteristics of the first image artifact being different than the second set of characteristics of the first image artifact and the third set of characteristics of the first image artifact. The second set of characteristics of the first image artifact being different than the third set of characteristics of the first image artifact. The second image artifact has a first set of characteristics under the first MR field strength, a second set of characteristics under the second MR field strength, and a third set of characteristics under the third MR field strength. The first set of characteristics of the second image artifact being different than the second set of characteristics of the second image artifact and the third set of characteristics of the second image artifact. The second set of characteristics of the second image artifact being different than the third set of characteristics of the second image artifact. The third image artifact has a first set of characteristics under the first MR field strength, a second set of characteristics under the second MR field strength, and a third set of characteristics under the third MR field strength. The first set of characteristics of the third image artifact being different than the second set of characteristics of the third image artifact and the third set of characteristics of the third image artifact. The second set of characteristics of the third image artifact being different than the third set of characteristics of the third image artifact.

An example method of performing an interventional medical treatment under MRI comprises positioning a patient within a MR scanner; scanning a first portion of the patient using the MR scanner; obtaining a MR image of the first portion of the patient; identifying a tissue that has predefined characteristics using the MR image; selecting a procedure to treat the tissue based upon the predefined characteristics; selecting MR sequences and/or parameters to use during performance of the selected procedure; selecting a medical device to accomplish performance of a medical procedure based upon the selected MR sequences and/or parameters intended to be used during performance of the selected procedure; while the patient remains positioned within the MR scanner used to scan a portion of the patient, advancing the medical device into a bodily passage and to the tissue while scanning a second portion of the patient that includes the medical device using the MR scanner; obtaining a MR image of the second portion of the patient that includes the medical device; confirming the position of the medical device within the bodily passage; and performing treatment using the medical device.

Additional understanding of these example medical systems, medical devices, kits, and methods can be obtained by review of the detailed description, below, and the appended drawings.

### BRIEF DESCRIPTION OF THE DRAWINGS

FIG. 1 is a partial elevation view of a first example medical system that includes a first example medical device and a second example medical device.

FIG. 2 is an elevation view of the first example medical device included in the medical system illustrated in FIG. 1.

FIG. 3 is an elevation view of the second example medical device included in the medical system illustrated in FIG. 1.

FIG. 4 is a magnified view of area A in FIG. 2.

FIG. 5 is a magnified view of area B in FIG. 3.

FIG. 6 is a cross-sectional view of the second medical device shown in FIG. 3 taken along line 6-6.

FIG. 7 is a graph illustrating the simulated magnetic susceptibility of some diamagnetic and paramagnetic metals and metal alloys.

FIG. 8 is a graph illustrating the simulated magnetization saturation for some ferromagnetic metals and metal alloys.

FIG. 9 is a simulated magnetic field perturbation and image artifact of a medical device with various metal alloy markers, including 304V cold-worked stainless steel.

FIG. 10 is a simulated magnetic field perturbation and image artifact of a medical device with various metal alloy markers, including 304V annealed stainless steel.

FIG. 11 is a partial perspective view of an example alternative medical device that can be included in a medical system.

FIG. 12 is a cross-sectional view of the medical device shown in FIG. 11 taken along line 12-12.

FIG. 13 is a cross-sectional view of another example alternative medical device that can be included in a medical system.

FIG. 14 is a cross-sectional view of another example alternative medical device that can be included in a medical system.

FIG. 15 is a partial perspective view of another example alternative medical device that can be included in a medical system.

FIG. 16 is a partial perspective view of another example alternative medical device that can be included in a medical system.

FIG. 17 is a magnified view of area C in FIG. 16.

FIG. 18 is a magnified view of area D in FIG. 17.

FIG. 19 is a magnified view of 304L plastic compounding fine powder.

FIG. 20 is a magnified view of 430L plastic compounding coarse powder with a low surface area.

FIG. 21 is magnified view of 430L plastic compounding coarse powder with a high surface area.

FIG. 22 illustrates a solid foil AD-MU-80 plug, an epoxy plug, a plug that includes SS 316 suspended in a bulk material (e.g. epoxy), and a plug that include SS 430L suspended in a bulk material (e.g., epoxy). Each plug is held in place using an epoxy.

FIG. 23 illustrates the plugs shown in FIG. 22 under gradient refocusing echo (GRE) MR imaging at 3.0 Tesla (T).

FIG. 24 illustrates the plugs shown in FIG. 22 under spin echo (SE) MR imaging at 3.0 T.

FIG. 25 is a partial perspective view of another example alternative medical device that can be included in a medical system.

FIG. 26 is an elevation view of a set of example alternative medical devices that can be included in a medical system.

FIG. 27 illustrates a control medical device and the medical devices shown in FIG. 26 in copper sulfate phantom solution under 0.55 T.

FIG. 28 illustrates a control medical device and the medical devices shown in FIG. 26 in copper sulfate phantom solution under 1.5 T.

FIG. 29 illustrates a control medical device and the medical devices shown in FIG. 26 in porcine tissue under 0.55 T.

FIG. 30 illustrates the medical devices shown in FIG. 26 in porcine tissue under 1.5 T.

FIG. 31 illustrates an example kit useful in performing treatment under MRI.

FIG. 32 is a schematic illustration of an example method of performing treatment under MRI.

FIG. 33 is another schematic illustration of an example method of performing treatment under MRI.

FIG. 34 is another schematic illustration of an example method of performing treatment under MRI.

### DETAILED DESCRIPTION OF SELECTED EXAMPLES

The following detailed description and the appended drawings describe and illustrate various example medical systems useful in performing treatment under MRI, medical devices useful in performing treatment under MRI, kits useful in performing treatment under MRI, and methods of performing interventional medical treatment under MRI. The description and illustration of these examples are provided to enable one skilled in the art to make and use a medical system, a medical device, a kit, and to practice a method of performing an interventional medical treatment under MRI. They are not intended to limit the scope of the invention, or the protection sought, in any manner. The invention is capable of being practiced or carried out in various ways and the examples described and illustrated herein are merely selected examples of the various ways of practicing or carrying out the invention and are not considered exhaustive.

As used herein, the term “attached” refers to one member being secured to another member such that the members do not completely separate from each other during use performed in accordance with the intended use of an item that includes the members in their attached form.

As used herein, the term “plug” refers to a member having a size and configuration suitable for disposition within a hole, passageway, recess, or void in another member. The term does not require any particular size or configuration, and the size and configuration of a particular plug will depend on the size and configuration of the hole, passageway, recess, or void into which the plug is intended to be disposed.

As used herein, the term “circumference” refers to an external, or internal, enclosing boundary of a body, element, or feature and does not impart any structural configuration on the body, element, or feature.

As used herein, the term “marker” refers to a discrete deposit of a first material on a second material such that the first material is visible under MRI and is distinguishable from the second material under MRI, a portion of an interventional device in which a first material has been incorporated into a second material such that the combination of the first and second materials is visible under MRI and is distinguishable from the second material under MRI, and/or a portion of an interventional device in which a material that forms a portion of an interventional device has been manipulated such that the portion is visible under MRI and is distinguishable from the remainder of the interventional device under MRI.

As used herein, the term “passive,” in relation to a marker, refers to a marker that is either unpowered or powered exclusively by the electromagnetic field of a MR scanner.

As used herein, the term “treatment” refers to a medical procedure performed on or in a portion of a body of a patient. Examples of treatments include delivery of an agent to a site within a body vessel, modification of a local environment inside of a body vessel such as by heating or cooling, and removal of a tissue or portion of a tissue from a site within a body of a patient (i.e., biopsy).

As used herein, the term “magnetic susceptibility” refers to the intrinsic property of a material that relates to how much the material will become magnetized in an applied magnetic field.

As used herein, the term “susceptibility,” without “magnetic,” refers to the ability of an element to influence an external magnetic field. Susceptibility is dependent on various properties of an element, including the size, density, volume, geometric configuration, and other physical properties, and the magnetic susceptibility of the material of which the element is formed.

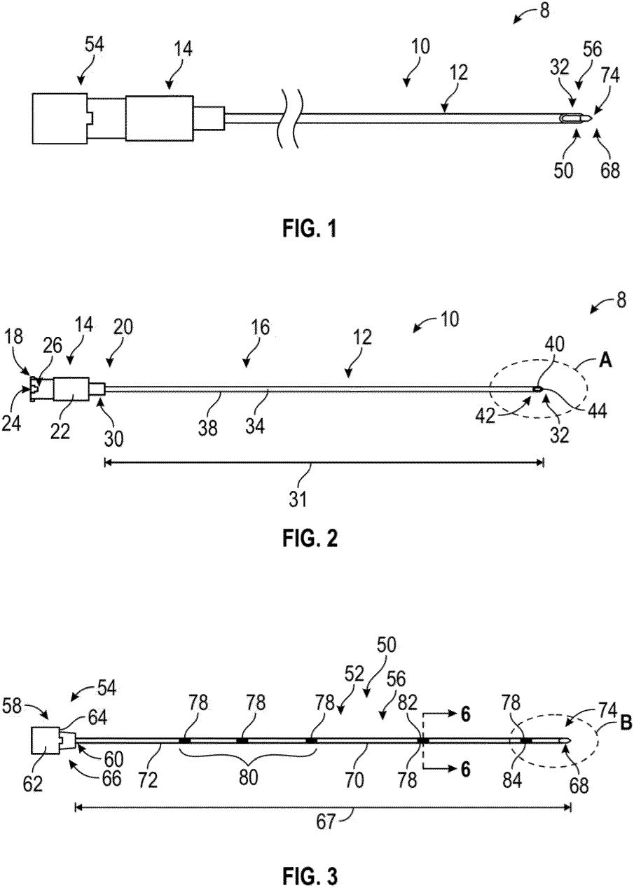

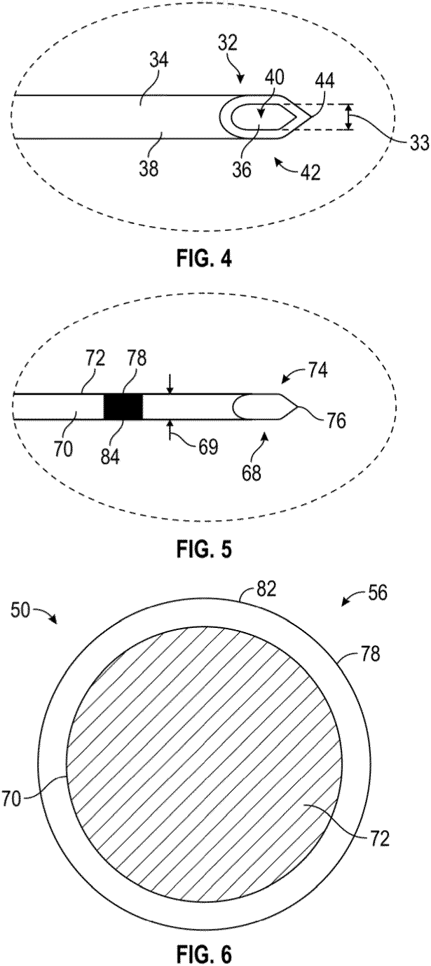

FIGS. 1, 2, 3, 4, 5, and 6 illustrate a first example medical system 8. In this example, the first example medical system 8 includes a first medical device 10 and a second medical device 50.

In the illustrated embodiment, the first medical device 10 is a cannula 12 and the second medical device 50 is a stylet 52 that can be used coaxially with the first medical device 10. The cannula 12 has a hub member 14 and an elongate member 16. The hub member 14 has a proximal end 18, a distal end 20, and a main body 22 that defines a lumen 24 and a projection 26. The hub member 14 provides a mechanism for attaching other devices to the cannula 12 (e.g., second medical device 50). The lumen 24 extends from the proximal end 18 to the distal end 20 such that one or more devices can be passed into, and through, the hub member 14. The elongate member 16 has a proximal end 30, a distal end 32, a length 31, an inside diameter 33, an outer surface 34, inner surface 36, and a main body 38 that defines a lumen 40 and a distal tapered tip 42 with a cutting edge 44. The lumen 40 extends from the proximal end 30 to the distal end 32 such that one or more devices can be passed into, and through, the elongate member 16 of the cannula 12. In the illustrated embodiment, the elongate member 16 of the cannula 12 is formed of a first material that has a first magnetic susceptibility.

The stylet 52 has a hub member 54 and an elongate member 56. The hub member 54 has a proximal end 58, a distal end 60, and a main body 62 that defines a recess 64. The hub member 54 provides a mechanism for attaching other devices to the stylet 52 (e.g., first medical device 10). The recess 64 extends into the main body 62 from a location between the proximal end 58 and the distal end 60 and toward the proximal end 58. The recess 64 is sized to receive the projection 26 defined by the hub member 14 of the cannula 12 such that releasable attachment between the cannula 12 and the stylet 52 can be accomplished. The elongate member 56 has a proximal end 66, a distal end 68, a length 67, an outside diameter 69, an outer surface 70, and a main body 72 that defines a solid member with a distal tapered tip 74 with a cutting edge 76. The length 67 of the elongate member 56 of the stylet 52 is greater than the length 31 of the elongate member 16 of the cannula 12. The outside diameter 69 of the elongate member 56 of the stylet 52 is less than the inside diameter 33 of the elongate member 16 of the cannula 12. This structural arrangement allows for the elongate member 56 of the stylet 52 to be passed into, and through, the lumen 24 of the hub member 14 and the lumen 40 of the elongate member 16 of the cannula 12, as shown in FIG. 1. The elongate member 56 of the stylet 52 is formed of a second material that has a second magnetic susceptibility. In the illustrated embodiment, the first material that forms the elongate member 16 of the cannula 12 and the second material that forms the elongate member 56 of the stylet 52 are the same.

The elongate member 56 of the stylet 52 includes a plurality of markers 78. Each marker of the plurality of markers is disposed around the entire circumference of the main body 72 of the elongate member 56 of the stylet 52. Each marker of the plurality of markers 78 is a band of material attached to the stylet 52 and is formed of a material that is different than the first and second materials and that has a magnetic susceptibility that is different than (e.g., is greater than, less than) the first magnetic susceptibility and the second magnetic susceptibility. In the illustrated embodiment, the first and second materials are non-magnetically susceptible materials, or are formed of a material with low magnetic susceptibility (e.g., a non-paramagnetic material, a non-ferromagnetic material, a non-diamagnetic material) relative to the magnetic susceptibility of a material that forms a marker included on a medical device, and the material used to form a marker of the plurality of markers 78 is a magnetically susceptible material, a material that has a high magnitude of magnetic susceptibility (e.g., a diamagnetic, a paramagnetic material, a ferromagnetic material) relative to the magnitude of magnetic susceptibility of a material that forms another portion of a medical device (e.g., elongate member). Therefore, the material forming a marker has a magnetic susceptibility that is different than (e.g., greater than, less than) the first magnetic susceptibility and the second magnetic susceptibility.

The plurality of markers 78 includes a first subset of markers 80, a second subset of markers 82, and a third subset of markers 84. Each marker in the first subset of markers 80 is disposed between the proximal end 66 and the distal end 68 of the elongate member 56 of the second medical device 50 and is formed of a third material that is different than the first and second materials and that has a third magnetic susceptibility that is different than (e.g., greater than, less than) the first magnetic susceptibility and the second magnetic susceptibility. Each marker in the second subset of markers 82 is disposed between the first subset of markers 80 and the third subset of markers 84 and is formed of a fourth material that is different than the first material, the second material, and the third material and that has a fourth magnetic susceptibility that is different than (e.g., greater than, less than) the first magnetic susceptibility and second magnetic susceptibility and different than (e.g., greater than, less than) the third magnetic susceptibility. However, alternative embodiments can include markers in which each marker in a second subset of markers is formed of a fourth material that is different than a first material, a second material, and is the same as a third material and/or that has a fourth magnetic susceptibility that is different than (e.g., greater than, less than) a first magnetic susceptibility and a second magnetic susceptibility and has the same magnetic susceptibility, the same susceptibility, or a different susceptibility, as a third magnetic susceptibility. Each marker in the third subset of markers 84 is disposed between the second subset of markers 82 and the distal end 68 of the elongate member 56 of the stylet 52 and is formed of a fifth material that is different than the first material, the second material, the third material, and the fourth material and that has a fifth magnetic susceptibility that is different than (e.g., greater than, less than) the first magnetic susceptibility and the second magnetic susceptibility and different than (e.g., greater than, less than) the third magnetic susceptibility and the fourth magnetic susceptibility. However, alternative embodiments can include markers in which each marker in a third subset of markers is formed of a fifth material that is different than a first material, a second material, and is the same as a third material and/or a fourth material and/or that has a fifth magnetic susceptibility that is different than (e.g., greater than, less than) a first magnetic susceptibility and a second magnetic susceptibility and has the same magnetic susceptibility, the same susceptibility, or a different susceptibility, as a third magnetic susceptibility and/or a fourth magnetic susceptibility. In the illustrated embodiment, the first subset of markers 80 includes three markers, the second subset of markers 82 includes one marker, and the third subset of markers 84 includes one marker. However, alternative embodiments can include any suitable number of markers in a subset of a plurality of markers. Alternatively, a medical device can include a plurality of markers that includes a first marker, a second marker, and a third marker. The first marker can create a first image artifact under a first field strength and/or a first MR sequence, the second marker can create a second image artifact under a second field strength and/or a second MR sequence, and the third marker can create a third image artifact under a third field strength and/or a third MR sequence. The first image artifact is different than the second image artifact and the third image artifact. The second image artifact is different than the third image artifact. The first MR field strength is different than the second MR field strength and the third MR field strength. The second MR field strength is different than the third MR field strength. The first image artifact has a first set of characteristics (e.g., size, shape, pattern, and/or intensity) under the first MR field strength, a second set of characteristics under the second MR field strength, and a third set of characteristics under the third MR field strength. The first set of characteristics of the first image artifact being different than the second set of characteristics of the first image artifact and the third set of characteristics of the first image artifact. The second set of characteristics of the first image artifact being different than the third set of characteristics of the first image artifact. The second image artifact has a first set of characteristics under the first MR field strength, a second set of characteristics under the second MR field strength, and a third set of characteristics under the third MR field strength. The first set of characteristics of the second image artifact being different than the second set of characteristics of the second image artifact and the third set of characteristics of the second image artifact. The second set of characteristics of the second image artifact being different than the third set of characteristics of the second image artifact. The third image artifact has a first set of characteristics under the first MR field strength, a second set of characteristics under the second MR field strength, and a third set of characteristics under the third MR field strength. The first set of characteristics of the third image artifact being different than the second set of characteristics of the third image artifact and the third set of characteristics of the third image artifact. The second set of characteristics of the third image artifact being different than the third set of characteristics of the third image artifact.

Alternatively, a medical system can include a first medical device and a second medical device. Each of the first and second medical devices include a plurality of markers that includes a first marker, a second marker, and a third marker. The first marker can create a first image artifact under a first field strength and/or a first MR sequence, the second marker can create a second image artifact under a second field strength and/or a second MR sequence, and the third marker can create a third image artifact under a third field strength and/or a third MR sequence. The first image artifact is different than the second image artifact and the third image artifact. The second image artifact is different than the third image artifact. Alternatively, overlapping of markers (e.g., first markers, second markers, third markers) can create an image artifact under a first, second, and/or third field strength.

The first material forming the elongate member 16 of the first medical device 10, the second material forming the elongate member 56 of the second medical device 50, and the material forming any marker included on a medical device, according to an embodiment, can comprise any suitable material having the magnetic susceptibility described herein. For example, a first material, a second material, a third material, a fourth material, and/or a fifth material can comprise any suitable MRI compatible material having the magnetic susceptibility described herein. In addition, or in an alternative embodiment, a first material, a second material, a third material, a fourth material, a fifth material, and/or any other material described herein can comprise any suitable MRI compatible material having a susceptibility, or relative susceptibility, similar to the magnetic susceptibility, or relative magnetic susceptibilities, described herein relative to the portion of a system or device which the material forms. In the embodiment shown, each of the first material and the second material comprises titanium, each marker in the first subset 80 of the plurality of markers 78 is formed of a stainless steel (e.g., 304V SS), each marker in the second subset 82 of the plurality of markers 78 is formed of a first alloy that includes less than 1% of iron by weight, and each marker in the third subset 84 of the plurality of markers 78 is formed of a second alloy, which can be the same or different than the first alloy (e.g., the second alloy has the same or different magnetic susceptibility as the first alloy, the second alloy has the same or different susceptibility as the first alloy), that includes less than 1% of iron by weight. In a more specific embodiment, each of the first material and the second material comprises titanium, each marker in the first subset 80 of the plurality of markers 78 is formed of stainless steel (e.g., 304V SS), each marker in the second subset 82 of the plurality of markers 78 is formed of a first cobalt-chromium alloy (e.g., FWM 1537 (Co-Cr)), and each marker in the third subset 84 of the plurality of markers 78 is formed of a second cobalt-chromium alloy (e.g., L605 (Co-Cr)) that is different than the first cobalt-chromium alloy. Use of these materials results in each marker of the plurality of markers 78 acting as a passive marker when using MRI to create a MR image.

While particular materials have been described as forming the elongate member 16 of the cannula 12, the elongate member 56 of the stylet 52, and each marker in the plurality of markers 78, a medical device, or a portion thereof (e.g., elongate member), and a marker can be formed of any suitable material having the magnetic susceptibility in accordance with the description herein. For example, a first medical device, a second medical device, and a marker included on a medical device can be formed of any suitable MRI compatible material having the magnetic susceptibility described herein. Selection of a suitable material can be based on various considerations, including the intended use of a first medical device, a second medical device, and/or a marker. Examples of materials considered suitable to form a first medical device, a second medical device, and/or a marker, or a portion thereof, include biocompatible materials, materials that can be made biocompatible, MRI compatible materials, metals, electrically insulating materials, electrically non-conducting materials, non-ferromagnetic materials, such as a non-magnetically susceptible materials, non-diamagnetic materials, passive materials, magnetically susceptible materials, including diamagnetic materials (e.g., pyrolytic carbon), paramagnetic materials, or ferromagnetic materials, ferromagnetic passive materials, Ferritic Stainless Steel, Ferritic Stainless Steel 430L powder, shape memory alloys, including nickel-titanium alloys, such as Nitinol, austentic nickel-chromium based alloys, such as Inconel (e.g., Inconel 625), a brand for a family of austenitic nickel-chromium-based superalloys from Special Metals Corporation, stainless steel, 304V SS, including Austenitic stainless steel, stainless steel containing Iron, stainless steel including Inconel, iron, cobalt, cobalt chromium, cobalt chromium alloys, titanium, materials (e.g., stainless steel) having a hardness of about 192 KSI, nickel, nickel plating, bright nickel plating, nickel strike plating, alloys that includes less than 1% of iron by weight, metals that includes murinite, such as MuMetal, which is a brand for a nickel-iron ferromagnetic alloy with a very high permeability from Magnetic Shield Corporation, plastics, polymers, PEEK, carbon-filled PEEK, polyethylene, such as high-density polyethylene (HDPE), polypropylene, polycarbonates, silicone, Delrin, ceramics, transparent materials, opaque materials, ceramics, the materials described herein, combinations of the described herein, and any other material considered suitable for a particular embodiment. A marker included in a medical device provides a mechanism for using the medical device under MRI such that the marker is a discrete indicator providing conspicuity on the portion of the medical device on which the marker is disposed (e.g., elongate member, tip). When more than one marker is included on a medical device, as described herein, a first marker can have a first conspicuity under MRI (e.g., create a first image artifact under MRI) and a second marker can have a second conspicuity under MRI (e.g., create a second image artifact under MRI) that is the same as, or different from, the first conspicuity depending on the material forming the first and second markers. This can be accomplished by forming the markers of the same, or different materials, and/or include a greater volume of one material relative to another.

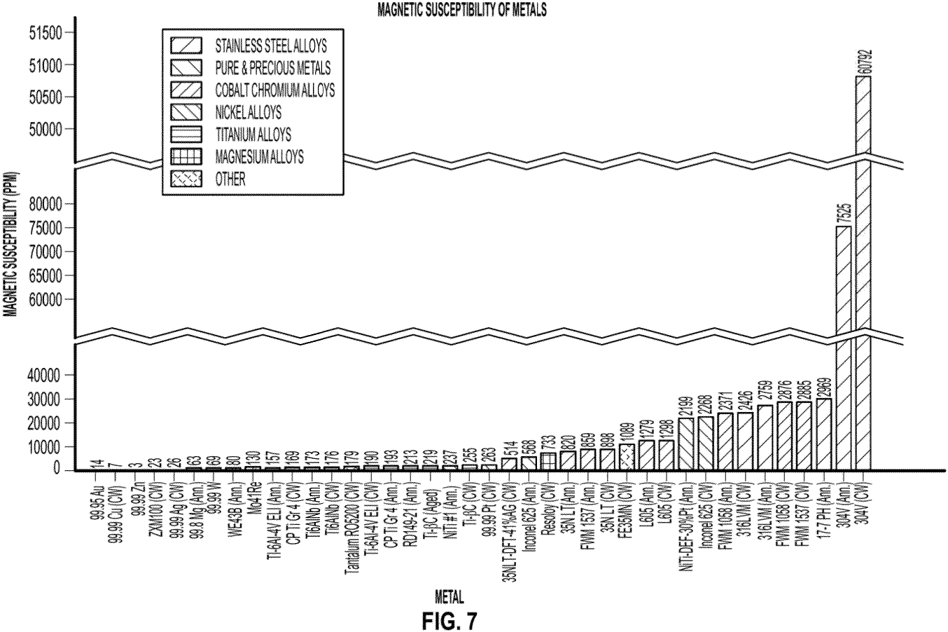

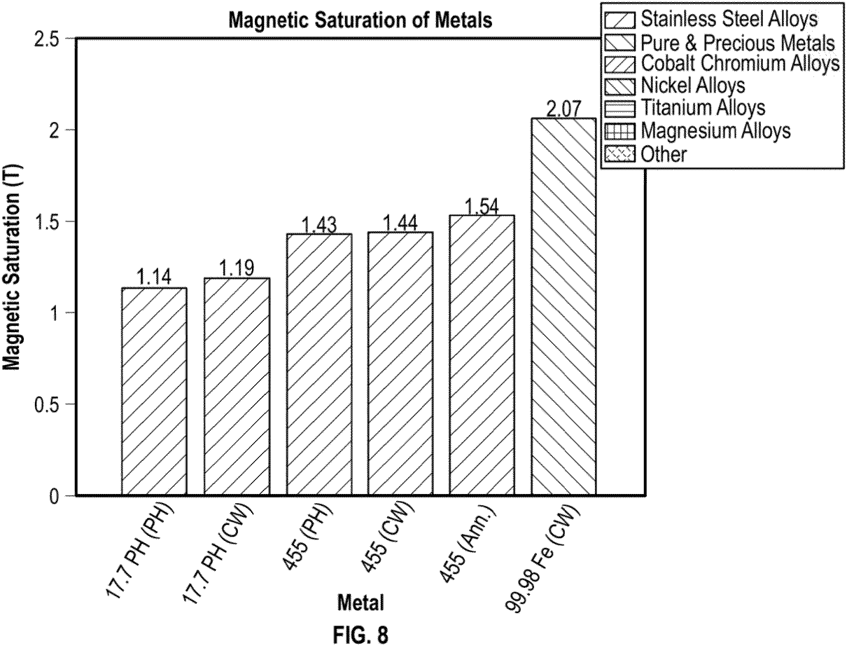

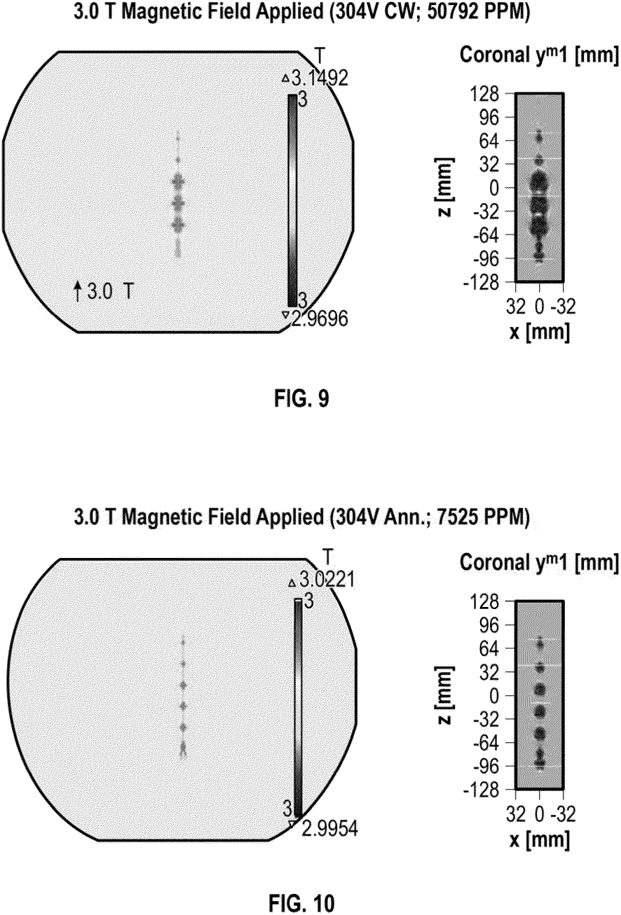

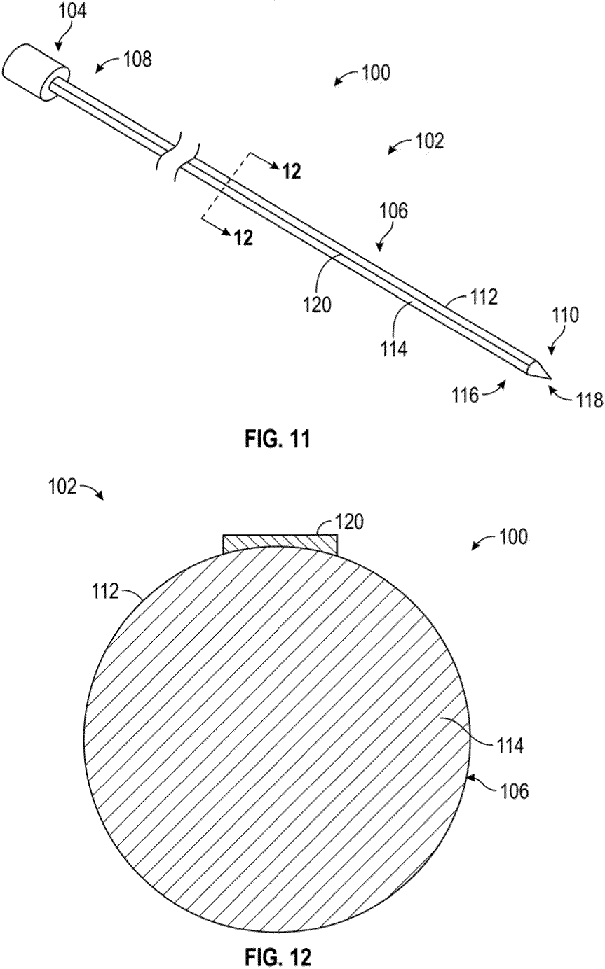

FIG. 7 is a graph illustrating the simulated magnetic susceptibility of some diamagnetic and paramagnetic metals and metal alloys that can be used to form a first medical device, a second medical device, and/or a marker. FIG. 8 is a graph illustrating the simulated magnetization saturation for some ferromagnetic metals and metal alloys that can be used to form a first medical device, a second medical device, and/or a marker. As described herein, a marker can be formed of any suitable material having the magnetic susceptibility described herein. FIG. 9 is a simulated magnetic field perturbation and image artifact of a medical device with various metal alloy markers, including 304V cold-worked stainless steel. FIG. 10 is a simulated magnetic field perturbation and image artifact of a medical device with various metal alloy markers, including 304V annealed stainless steel.

A medical system, such as those described herein, can include any suitable number and type of medical devices. For example, a first medical device can comprise any suitable type of cannula having any suitable length and gauge and a second medical device can comprise any suitable type of stylet having any suitable length and gauge. For example, a first medical device can comprise an Inconel Chiba Needle, an Inconel micropuncture needle, or any other needle considered suitable for a particular embodiment, have a length equal to, greater then, or less than 7 centimeters, 15 centimeters, or any other length considered suitable for a particular embodiment, and/or have a gauge equal to, greater than, or less than 20, 21, 22, or any other gauge considered suitable for a particular embodiment. Any of the medical systems and/or medical devices described herein can be utilized individually, or in combination with, another medical system and/or device.

Any suitable number of markers can be positioned on a medical device according to an embodiment. While the second medical device 50 includes five markers 78, it is to be appreciated that a medical device according to an embodiment can include any number of markers considered suitable for the intended use of the particular medical device and each marker can be positioned at any suitable location on any suitable component of the medical device. Examples of suitable numbers of markers for inclusion in a medical device according to an embodiment include one marker, two markers, more than two markers, three markers, a plurality of markers, four markers, five markers, six markers, seven markers, eight markers, nine markers, ten markers, and more than ten markers. Furthermore, in embodiments that include two or more markers, the markers can be spaced from each other by any desired distance. It should be noted, though, that, because the markers produce visual artifacts and their utility in the medical device is based on this production of visual artifacts in MRI procedures, it is desirable to space markers from each other on a medical device by a distance that does not result in overlapping or nearly overlapping visual artifacts. However, alternative embodiments can include markers that are spaced from each other by a distance that does produce overlapping visual artifacts. For example, overlapping of visual artifacts may occur under one type of MR image sequence (e.g., gradient refocusing echo) and may not occur under another type of MR image sequence (e.g., spin echo).

While each marker of the plurality of markers 78 has been illustrated as a marker band (e.g., circumferential marker) disposed on the outer surface 70 of the elongate member 56 of the stylet 52, a marker can comprise any suitable structure attached to a medical device (e.g., stylet, cannula) or any suitable treatment imparted on a medical device. For example, a marker can have any suitable shape and include bands of material, magnetic inks, sputtered magnetite, dimpling, swagging, or peening the material that forms a portion of a medical device (e.g., annealed 304 stainless steel), and/or a single layer or multiple layers of a material (e.g., metal, metal alloy), or materials (e.g., metals, metal alloys), deposited along an outer surface and/or an inner surface of a medical device (e.g., elongate member of a medical device) to form distinct markers visible under MRI. Any suitable marker can be included in a medical device, such as those described herein. Examples of markers considered suitable to include in a medical device are also described in U.S. Pat. Application No.: 16/454,905, filed on Jun. 27, 2019, which is hereby incorporated by reference in its entirety for the purpose of describing markers considered suitable to include in a medical device. Each marker of the plurality of markers 78 can have any suitable configuration. As shown in FIG. 6, circumferential markers can be disposed around the entire circumference of the main body 72 of the elongate member 56 of the stylet 52. Alternatively, a marker that defines a partial circumference can be disposed around a main body of a portion of a medical device (e.g., elongate member of a stylet, elongate member of a cannula) according to an embodiment such that the marker extends around only a portion of the circumference of the main body. Alternatively, a marker can comprise a structure that defines a curve along a portion of a length of an elongate member, a structure that defines any suitable geometric shape on a portion of an elongate member (e.g., circle, polygon, rectangle), a structure that defines a spiral along a portion of a length of an elongate member, or any combination thereof. The marker configurations described herein provide a mechanism for creating a marker that creates an image artifact under MRI that is distinguishable from another medical device, or combination of medical devices, the surrounding fluid, and/or tissue being imaged under MRI.

While the elongate member 16 of the cannula 12 has been illustrated as including a distal tapered tip 42 with a cutting edge 44 and the elongate member 56 of the stylet 52 has been illustrated as including a distal tapered tip 74 with a cutting edge 76, an elongate member included on a medical device can include any suitable structural configuration, such as those with blunted, or non-tapered, distal tips. Furthermore, in alternative embodiments a hub member can include any suitable structure to accomplish an attachment to another device and/or be omitted from a medical device and/or a stylet can be hollow such that it defines a lumen that extends the entire length of the stylet or a portion of the length of the stylet.

The medical systems and/or medical devices described herein can be used with any suitable number of outer sheaths to accomplish treatment. Selection of a suitable number of outer sheaths to utilize when completing sequential dilation, for example, can be based on the bodily passage within which a sequential dilation is being completed. Examples of numbers of outer sheaths considered suitable to utilize when completing sequential dilation include one, two, a plurality, three, four, more than four, and any other number considered suitable for a particular embodiment.

FIGS. 11 and 12 illustrate an example alternative medical device 100 that can be included in a medical system. In this example, the medical device 100 is an alternative embodiment of a stylet 102 that can be included in a medical system. The stylet 102 is similar to the stylet 50 illustrated in FIGS. 1, 3, 5, and 6 and described above, except as detailed below. In the illustrated embodiment, the stylet 102 has a hub member 104 and an elongate member 106. The elongate member 106 has a proximal end 108, a distal end 110, an outer surface 112, and a main body 114 that defines a solid member with a distal tapered tip 116 with a cutting edge 118.

The elongate member 106 includes a single marker 120 that extends along the entire length of the elongate member 106. In the embodiment shown, the marker 120 is formed as a single layer of a material (e.g., metal, metal alloy) deposited along the outer surface 112 of the elongate member 106 to form a distinct marker visible under MRI. The elongate member 106 is formed of a third material and the marker 120 is formed of a fourth material. The third material has a third magnetic susceptibility and the fourth material has a fourth magnetic susceptibility that is different than (e.g., greater than, less than) the third magnetic susceptibility. While the marker 120 has been illustrated as extending along the entire length of the elongate member 106, a marker included on an elongate member can alternatively extend along a portion of the length of an elongate member.

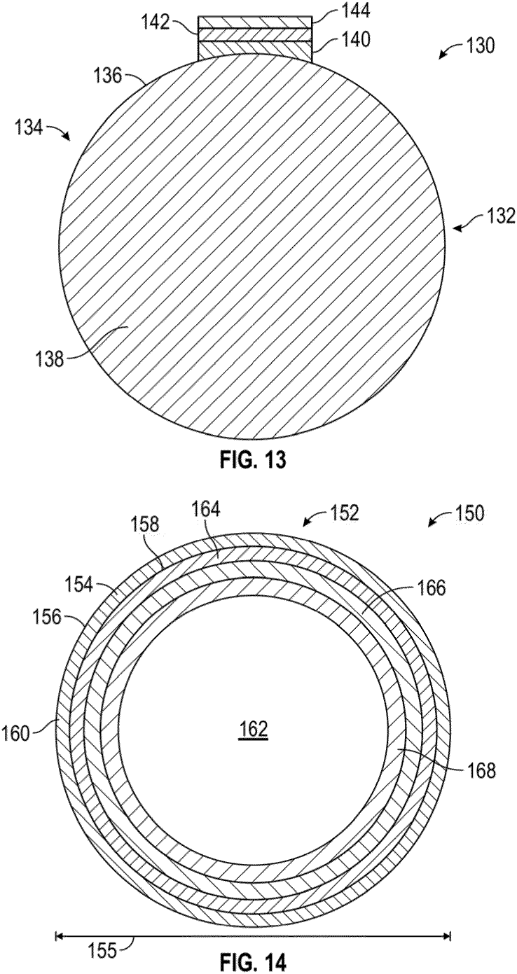

FIG. 13 illustrates another example alternative medical device 130 that can be included in a medical system. In this example, the medical device 130 is an alternative embodiment of a stylet 132 that can be included in a medical system. The stylet 132 is similar to the stylet 100 illustrated in FIGS. 11 and 12 and described above, except as detailed below. In the illustrated embodiment, the stylet 132 has an elongate member 134 that has an outer surface 136, and a main body 138.

The elongate member 134 includes a first marker 140, a second marker 142, and a third marker 144. Each of the markers 140, 142, 144 extends along the entire length of the elongate member 134. The first marker 140 is formed as a first layer of material disposed on the outer surface 136 of the elongate member 134, a second marker 142 is formed as a second layer of material disposed on the first marker 140, and a third marker 144 is formed as a third layer of material disposed on the second marker 142. In this embodiment, the markers 140, 142, 144 are disposed axially along a portion of the outer surface 136 of the elongate member 134 as deposited layers of material to form distinct markers visible under MRI (e.g., along a portion of axial length and/or at discrete points). While each of the markers 140, 142, 144 has been illustrated as extending along the entire length of the elongate member 134, a marker included on an elongate member can alternatively extend along a portion of the length of an elongate member.

The materials forming the first marker 140, the second marker 142, and the third marker 144 can comprise any suitable material having the magnetic susceptibility described herein. The material forming the first marker 140 can be the same as the material forming the second marker 142 and the material forming the third marker 144. Alternatively, the material forming the first marker 140 can be the different than the material forming the second marker 142 and the material forming the third marker 144. Alternatively, the material forming the first marker 140 can be the same as the material forming the second marker 142 and different than the material forming the third marker 144. Alternatively, the material forming the first marker 140 can be different than the material forming the second marker 142 and the same as the material forming the third marker 144. Any suitable configuration of materials having the magnetic susceptibilities described herein can be utilized to form distinct markers visible under MRI.

In the illustrated embodiment, the elongate member 134 is formed of a third material, the first marker 140 is formed of a fourth material, the second marker 142 is formed of a fifth material, and the sixth marker 144 is formed of a sixth material. The third material has a third magnetic susceptibility, the fourth material has a fourth magnetic susceptibility, the fifth material has a fifth magnetic susceptibility, and the sixth material has a sixth magnetic susceptibility. The fourth magnetic susceptibility is different than (e.g., greater than, less than) the third magnetic susceptibility and different than, greater than, less then, or equal to the fifth magnetic susceptibility and/or the sixth magnetic susceptibility. The fifth magnetic susceptibility is different than (e.g., greater than, less than) the third magnetic susceptibility and different than, greater than, less then, or equal to the fourth magnetic susceptibility and/or the sixth magnetic susceptibility. The sixth magnetic susceptibility is different than (e.g., greater than, less than) the third magnetic susceptibility and different than, greater than, less then, or equal to the fourth magnetic susceptibility and/or the fifth magnetic susceptibility.

FIG. 14 illustrates another example alternative medical device 150 that can be included in a medical system. In this example, the medical device 150 is an alternative embodiment of a stylet 152 that can be included in a medical system. The stylet 152 is similar to the stylet 100 illustrated in FIGS. 11 and 12 and described above, except as detailed below. In the illustrated embodiment, the stylet 152 has an elongate member 154 that has an outside diameter 155, an outer surface 156, an inner surface 158, and a main body 160 that defines a lumen 162 that can extend through the entire length of the elongate member 154 or a portion of the length of the elongate member 154. A hub member included on a stylet similar to that illustrated in FIG. 11 can be included on stylet 152 and define a lumen that extends through the entire length of the hub member and provides access to the lumen 162 defined by the elongate member 154.

The elongate member 154 includes a first marker 164, a second marker 166, and a third marker 168. The first marker 164 extends along the entire circumference of the inner surface 158 of the elongate member 154, the second marker 166 extends along the entire circumference the first marker 164, the third marker 168 extends along the entire circumference of the second marker 166, and each of the markers 164, 166, 168 extends along a portion, or the entirety, of the length of the elongate member 154. In the embodiment shown, the first marker 164 is formed as a first layer of material disposed on the inner surface 158 of the elongate member 154, the second marker 166 is formed as a second layer of material disposed on the first marker 164, and the third marker 168 is formed as a layer of material disposed on the second marker 166. Each of the markers 164, 166, 168 comprises a deposited layer of material that forms a distinct marker visible under MRI.

The materials forming the first marker 164, the second marker 166, and the third marker 168 can comprise any suitable material having the magnetic susceptibility described herein. The material forming the first marker 164 can be the same as the material forming the second marker 166 and the material forming the third marker 168. Alternatively, the material forming the first marker 164 can be the different than the material forming the second marker 166 and the material forming the third marker 168. Alternatively, the material forming the first marker 164 can be the same as the material forming the second marker 166 and different than the material forming the third marker 168. Alternatively, the material forming the first marker 164 can be different than the material forming the second marker 166 and the same as the material forming the third marker 168. Any suitable configurations of materials having the magnetic susceptibilities described herein can be utilized to form distinct markers visible under MRI.

In the illustrated embodiment, the elongate member 154 is formed of a third material, the first marker 164 is formed of a fourth material, the second marker 166 is formed of a fifth material, and the sixth marker 168 is formed of a sixth material. The third material has a third magnetic susceptibility, the fourth material has a fourth magnetic susceptibility, the fifth material has a fifth magnetic susceptibility, and the sixth material has a sixth magnetic susceptibility. The fourth magnetic susceptibility is different than (e.g., greater than, less than) the third magnetic susceptibility and different than, greater than, less then, or equal to the fifth magnetic susceptibility and/or the sixth magnetic susceptibility. The fifth magnetic susceptibility is different than (e.g., greater than, less than) the third magnetic susceptibility and different than, greater than, less then, or equal to the fourth magnetic susceptibility and/or the sixth magnetic susceptibility. The sixth magnetic susceptibility is different than (e.g., greater than, less than) the third magnetic susceptibility and different than, greater than, less then, or equal to the fourth magnetic susceptibility and/or the fifth magnetic susceptibility.

While the markers 164, 166, and 168 are shown as multiple layers, a single marker can be utilized and disposed on an inner surface of a medical device and along the entire, or a portion of, the circumference and/or length of the medical device. In embodiments in which materials are layered and distinct materials are utilized for each layer, these structural arrangements can be used to exploit the effects each material has collectively on distorting the external magnetic field (e.g., creating image artifacts) when the medical device on which the materials are disposed is being visualized under MRI allowing for the tailoring of an image artifact anywhere on the device.

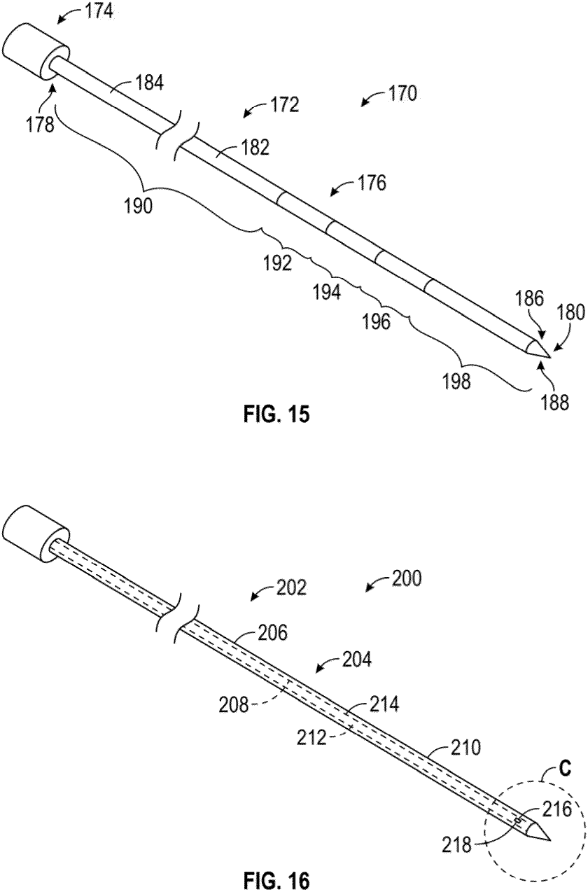

FIG. 15 illustrates another example alternative medical device 170 that can be included in a medical system. In this example, the sixth medical device 170 is an alternative embodiment of a stylet 172 that can be included in a medical system. The stylet 172 is similar to the stylet 10 illustrated in FIGS. 1, 3, 5, and 6 and described above, except as detailed below. In the illustrated embodiment, the stylet 172 has a hub member 174 and an elongate member 176. The elongate member 176 has a proximal end 178, a distal end 180, an outer surface 182, and a main body 184 that defines a solid member with a distal tapered tip 186 with a cutting edge 188.

The elongate member 176 includes a first portion 190, a second portion 192, a third portion 194, a fourth portion 196, and a fifth portion 198. The first portion 190 extends from the proximal end 178 of the elongate member 176 to the second portion 192. The second portion 192 extends from the first portion 190 to the third portion 194. The third portion 194 extends from the second portion 192 to the fourth portion 196. The fourth portion 196 extends from the third portion 194 to the fifth portion 198. The fifth portion 198 extends from the fourth portion 196 to the distal end 180 of the elongate member 176.

The first portion 190 is formed of a first material, the second portion 192 is formed of a second material, the third portion 194 is formed of a third material, the fourth portion 196 is formed of a fourth material, and the fifth portion 198 is formed of a fifth material. Any suitable material having the magnetic susceptibility described herein can be used to form each portion of the elongate member 176 (e.g., metal, polymer). For example, the material forming the portions 190, 192, 194, 196, 198 can be the same, or distinct from one another (e.g., distinct material properties). In embodiments in which the portions 190, 192, 194, 196, 198 are formed of one or more distinct materials, each portion can be joined to an adjacent portion using any suitable method or technique (e.g., welding, fusing, using an adhesive). Alternatively, a first set of the portions 190, 192, 194, 196, 198 of the elongate member 176 can be formed of the same material and a second set of the portions 190, 192, 194, 196, 198 of the elongate member 176 can be formed of a different material. In embodiments in which the portions 190, 192, 194, 196, 198 are formed of the same material, the material forming one or more portions, or each portion, of the elongate member 176 can be locally altered to change its magnetic properties (e.g., work hardened, tempered). When the portions 190, 192, 194, 196, 198 are formed from more than one material (e.g., are formed of distinct materials), or are formed of the same material but have distinct magnetic susceptibilities, or relative susceptibility, at least one portion can have a magnetic susceptibility that distorts an external magnetic field to create an image artifact. In these embodiments, the materials forming the portions 190, 192, 194, 196, 198 can be chosen such that their effects act independently or collectively on an external magnetic field to create an image artifact under MRI. This can be accomplished by forming one or more of the portions 190, 192, 194, 196, 198 as a marker having the magnetic susceptibility described herein.

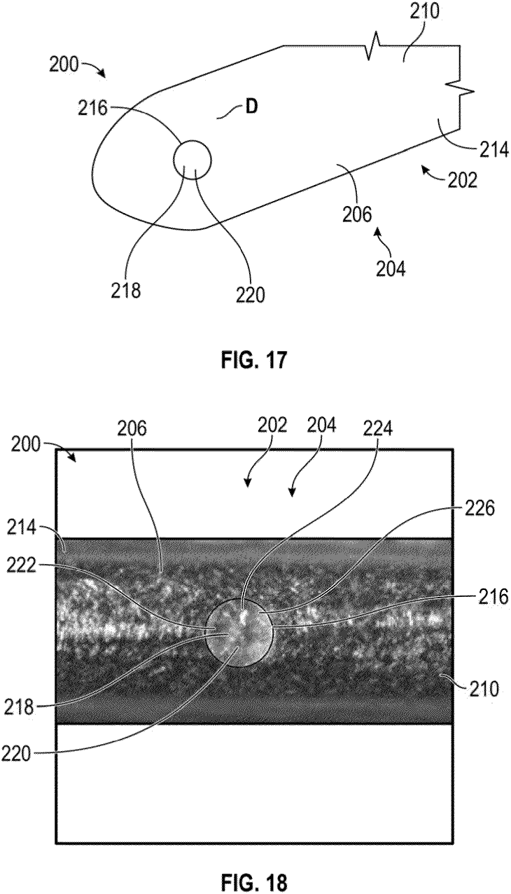

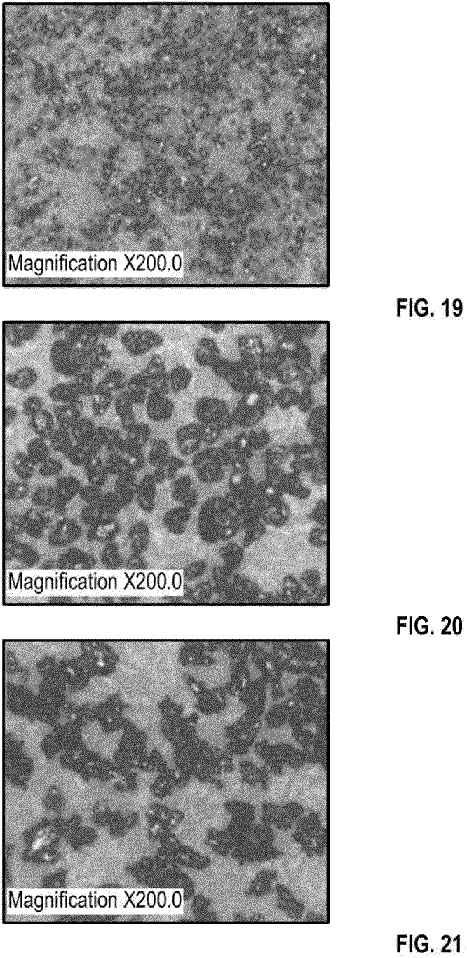

FIGS. 16, 17, and 18 illustrate another example alternative medical device 200 that can be included in a medical system. In this example, the medical device 200 is an alternative embodiment of a stylet 202 that can be included in a medical system. The stylet 202 is similar to the stylet 50 illustrated in FIGS. 1, 3, 5, and 6 and described above, except as detailed below. In the illustrated embodiment, the stylet 202 has an elongate member 204 that has an outer surface 206, an inner surface 208, and a main body 210 that defines a lumen 212 that can extend through the entire length of the elongate member 204 or a portion of the length of the elongate member 204.

The main body 210 of the stylet 202 defines a wall 214 that has the outer surface 206 and the inner surface 208. A wall thickness extends between the outer surface 206 and the inner surface 208. The main body 210 of the stylet 202 defines a passageway 216 that extends through the entire thickness of the wall 214 and from the inner surface 208 to the outer surface 206. Alternatively, a recess can extend only partially through the thickness of a wall, either from an inner surface toward an outer surface or from an outer surface toward an inner surface. A passageway and/or a recess included on a medical device can have any suitable dimensions and be positioned at any suitable location on a medical device. Examples of diameters considered suitable for a passageway and/or recess include diameters equal to, greater than, less than, or about 0.010 inches, 0.018 inches, 0.025 inches, between about 0.010 inches and about 0.025 inches, and any other diameter considered suitable for a particular embodiment.

In the embodiment illustrated, the elongate member 204 of the stylet 202 includes a single marker 218, which is a plug 220 disposed within the passageway 216. The plug 220 has a main body 222 that is a solid member that prevents access to the lumen 212 defined by the stylet 202. Alternative embodiments, however, can include more than one marker and/or a plug that defines a passageway that extends through the plug. The marker 218 is flush with the inner surface 208 and the outer surface 206 and, in the embodiment illustrated, has been pressed into the passageway 216. Additional securement can be used, if desired. For example, a marker can be laser welded to a wall of a stylet to secure the marker in a passageway. A plug 220 can have any suitable diameter and shape, which can be based on the diameter of a passageway within which a plug is intended to be disposed. Examples of diameters of plugs considered suitable to include in a medical device include diameters equal to, less than, greater than, or about 0.010 inches, 0.018 inches, 0.025 inches, between about 0.010 inches and about 0.025 inches, and any other diameter considered suitable for a particular embodiment.

A passageway and/or recess can be created in a medical device using any suitable technique or method, such as laser drilling, electric discharge machining (EDM), grinding, stamping, or microdrilling. A marker included on a medical device can be attached to the medical device using any suitable technique or method. Examples of techniques and method considered suitable to attach a marker to a medical device include pressing, welding, using adhesives, and any other technique or method considered suitable for a particular embodiment. For example, a marker can be inserted into, and attached to a material intended to form a medical device, or a portion of a medical device (e.g., an elongate member of a medical device), while the material forming the medical device is in its base form (e.g., an elongate member is in ribbon form). Subsequently, the base material (e.g., ribbon), which includes the marker, can be formed into the medical device, or portion of the medical device (e.g., elongate member), using any suitable method or technique (e.g., welding, drawing material).

In the embodiment illustrated, as shown in FIGS. 16, 17, and 18, the marker 218 (e.g., plug 220) comprises magnetically susceptible particles 224 (e.g., magnetically susceptible metallic particles) suspended within a bulk material 226 (e.g., epoxy resin). The elongate member 204 is formed of a third material that has a third magnetic susceptibility, the magnetically susceptible particles 224 are formed of a fourth material that has a fourth magnetic susceptibility, and the bulk material 226 is formed of a fifth material that has a fifth magnetic susceptibility. The fourth magnetic susceptibility is different than, equal to, less than, or greater than the third magnetic susceptibility. The fifth magnetic susceptibility is different than (e.g., greater than, less than) the third magnetic susceptibility and different than, equal to, less than, or greater than the fourth magnetic susceptibility.

The magnetically susceptible particles 224 can be used to distort a magnetic field used during MRI in order to generate tunable image artifacts in the resulting images. An alternative embodiment, however, can include a plug that is formed of a magnetically susceptible metal. Forming the marker 218 of magnetically susceptible particles 224 results in a medical device 200 that includes a passive marker that can be viewed when using MRI to create a MR image. The marker 218 has properties that produce visual image artifacts during MRI procedures in which the medical device 200 is imaged. These visual image artifacts can be used to determine placement of the medical device 200 relative to other portions of an image created under MRI, such as portions of a bodily passage into which the medical device 200 has been advanced and/or another medical device.

The magnetically susceptible particles 224 included in a plug can comprise any suitable type and size of magnetically susceptible metal particles having the magnetic susceptibility described herein. Examples of magnetically susceptible particles (e.g., magnetically susceptible metallic particles) considered suitable to include in a plug include MRI compatible materials, stainless steel, hardened forms stainless steel, ferritic series stainless steel (e.g., 400 series, such as 430L), stainless steel martensitic alloys (e.g., 316L, 304, 304L), nickel alloys, such as those used in magnetic shielding (e.g., AD-MU-80, Mu-Metal), the materials described herein, and any other material considered suitable for a particular embodiment. FIGS. 19, 20, and 21 illustrate various materials that can be used to create a plug for inclusion in a medical device, as described herein.

While the magnetically susceptible particles 224 have been illustrated as being suspended in a bulk material 226, magnetically susceptible particles of a marker can be suspended in any suitable MRI compatible material, such as those described herein. In addition, other non-susceptible materials that can be used to suspend magnetically susceptible particles, or to include in a suspension with magnetically susceptible particles, include polymers, thermoplastic polymers, such as nylon, Pebax, polyolefins, PEEL, FEP, or TFE, photopolymers, glass, ceramic microspheres, ceramic nanospheres, adhesive binders, MR-inert materials, the materials described herein, and any other material considered suitable for a particular embodiment. These materials can be added to improve even distribution of susceptible particles within a given volume and/or aid in the workability and manufacturability of a marker without directly affecting the resulting image artifact.

Use of plugs that include magnetically susceptible particles (e.g., metallic particles) suspended in a material that is not magnetically susceptible (e.g., epoxy resin), or that has a magnetic susceptibility that is different than (e.g., less than) the magnetic susceptibility of the magnetically susceptible particles, are considered advantageous at least because testing in imaging phantoms and tissue samples have demonstrated excellent marker visibility in multiple MR fields strength, imaging sequences, and machines and can be used to generate tunable susceptibility-based image artifacts under MR imaging. For example, these markers are tunable, allow for variable placement, and ease in manufacturability. The presence and manipulation of susceptibility markers along the shaft of an interventional device can improve safety, effectiveness, and efficiency of interventional MRI procedures.

While the marker 218 has been illustrated as disposed within a passageway defined by an elongate member of a medical device, alternative embodiments can position magnetically susceptible particles (e.g., metallic particles) suspended in a material that is not magnetically susceptible (e.g., epoxy resin), or that has a magnetic susceptibility that is different than (e.g., less than) the magnetic susceptibility of the magnetically susceptible particles, within a recess, or other feature, defined by a medical device (e.g., on an inner surface or outer surface of the medical device). A recess, or other features of a medical device, can be created on a medical device using any suitable technique or method, such as EDM, micro-machining, laser machining, grinding, and/or stamping.

A plug included in a medical device, such as those described herein, can be formed by potting particles using a two-part epoxy resin (e.g., medical grade epoxy resin, Loctite EA M-31CL), or any other suitable technique or method. Any suitable concentration of magnetically susceptible particles (e.g., metallic particles) can be used relative to the material within which the magnetically susceptible particles are suspended. For example, concentrations can range from between about 3 parts magnetically susceptible particles to about 1 part material that is not magnetically susceptible (e.g., epoxy resin), or that has a magnetic susceptibility that is different than (e.g., less than) the magnetic susceptibility of the magnetically susceptible particles, by mass and about 1 part magnetically susceptible particles to about 4 parts material that is not magnetically susceptible (e.g., epoxy resin), or that has a magnetic susceptibility that is different than (e.g., less than) the magnetic susceptibility of the magnetically susceptible particles, by mass. Alternatively, the magnetically susceptible particles included in a marker can include concentrations of a fraction of a percent (e.g., about 0.01% to about 0.99%) to a few percentage points (e.g., 5%) by weight relative to a material that is not magnetically susceptible (e.g., epoxy resin), or that has a magnetic susceptibility that is different than (e.g., less than) the magnetic susceptibility of the magnetically susceptible particles.

In alternative embodiments, solid pieces of material described as forming the magnetically susceptible particles can be cut and used as a plug and attached to an elongate member of a medical device as described herein. Alternatively, a solid piece of material (e.g., 0.002 inches thick AD-MU-80) can be die cut to diameters slightly smaller than a diameter of a passageway within which it is intended to be disposed. Subsequently, the solid piece of material can be bonded within a passageway defined by an elongate member of a medical device using a magnetically susceptible particle bulk material combination, or other materials, as described herein.

As described herein, the placement of a plurality of markers on a medical device can vary. However, it is considered advantageous to position each marker such that each image artifact created under MRI is clearly distinguishable from other image artifacts (e.g., using about a 1.5 centimeter gap between the boundaries of the artifacts generated). Optionally, a plurality of markers can be arranged in specific patterns to aid in identification and determining orientation of a medical device during use.,

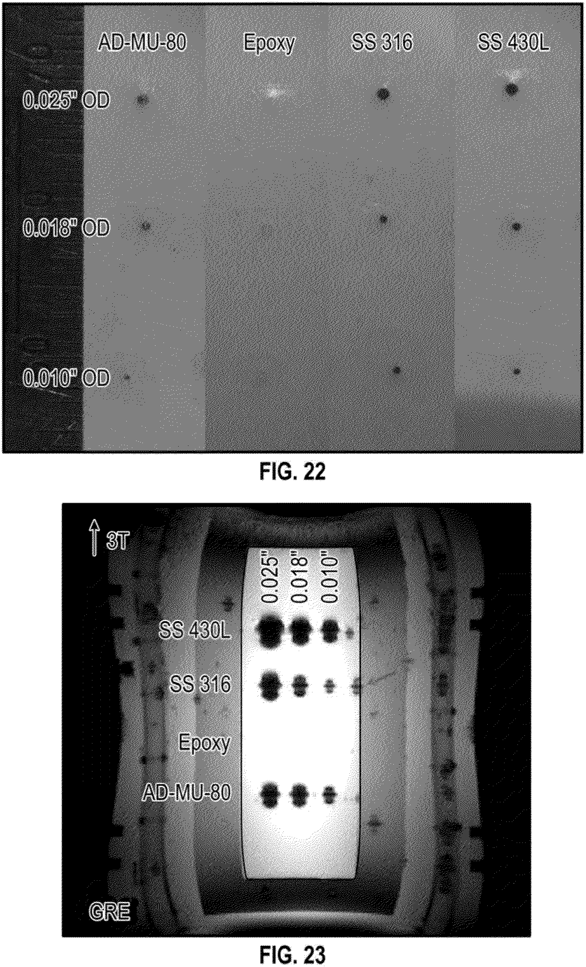

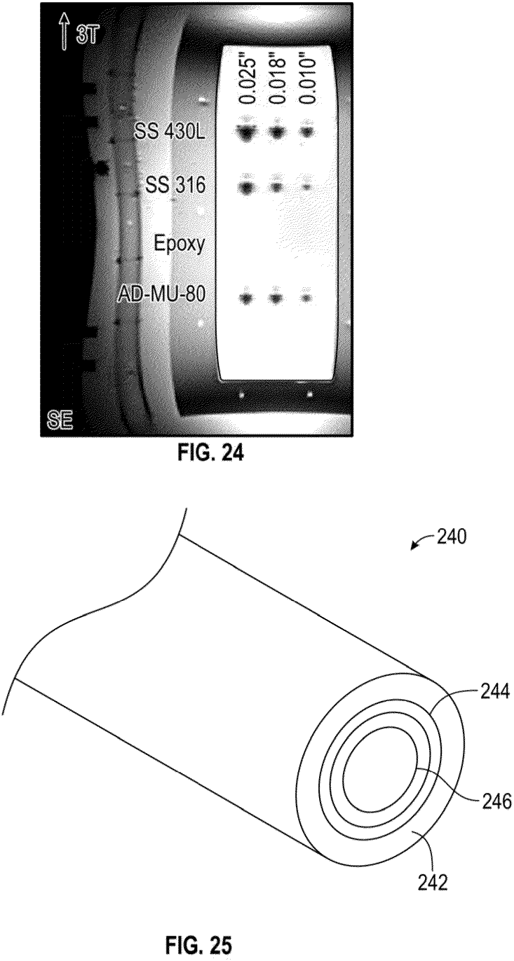

FIGS. 22, 23, and 24 illustrate marker plugs of varying diameters and materials cast separate from a medical device. FIGS. 23 and 24 illustrated these plugs being imaged under a magnetic field strength of 3.0 T. These figures illustrate the effects of the magnetically susceptible metallic particles suspended in an epoxy resin and measure artifact size. As shown, distinct marker artifacts have been demonstrated under multiple imaging modalities, including spin echo (SE) and gradient refocusing echo (GRE) sequences. In addition, distinct marker artifacts have also been demonstrated under other magnetic field strengths (e.g., 0.5 T, 1.5 T) and imaging modalities, such as high frame rate functional imaging sequences, true fast imaging with steady-state precession (TrueFisp), and fast low flip angle shot (FLAS) spoiled gradient-echos.

Optionally, a marker (e.g. a plug), such as those described herein, can be sealed (e.g., within a recess or passageway within which it is disposed) to increase biocompatibility, safety, and/or usability. For example, over coating of non-magnetically susceptible materials, such as non-magnetically susceptible metallic materials (e.g., gold, silver, or other alloys), inert materials, and/or non-metallic materials (e.g., fluoropolymers, ceramic Alumina) can be deposited, layered, or positioned over a portion of a medical device that includes a marker (e.g., plug) or the entirety of a medical device. Such over coating can be accomplished using any suitable technique or method, such as electroplating, sputtering, or vapor deposition.

In an alternative embodiment, a material can be compounded with magnetically susceptible particles (e.g., metallic particles) and can be insert or injection molded into a passageway or recess defined by a medical device. For example, magnetically susceptible particles and/or non-susceptible particles used as filler, such as those described herein, can be suspended in a material that is not an epoxy resin (e.g., material that is not magnetically susceptible, material that has a magnetic susceptibility that is different than (e.g., less than) the magnetic susceptibility of the material that forms the magnetically susceptible particles, adhesive binder). This can be accomplished by particle sintering and/or doping of a base material (e.g., polymer) with magnetically susceptible particles (e.g., metallic particles) and/or non-susceptible particles, which can be utilized to create a marker. A marker created using these materials can be used to create any suitable type of structure and/or marker, such as those described herein (e.g., bands, curves, stripes, coverings, plugs, coatings, extruded solids, elongate members, elongate members that defines a lumen, molded components). Creation of a structure and/or marker using these materials is considered advantageous at least because they can selectively enhance the visibility of at least part of an interventional device and/or introduce easily identifiable features under MRI guidance by generating tunable susceptibility artifacts.

For example, in one alternative embodiment, at least part of a medical device (e.g., a solid polymer stylet) can be doped with magnetically susceptible particles or solids to accentuate a tip of the medical device (e.g., stylet) or other portions of the elongate member of the medical device (e.g., stylet). In another alternative embodiment, portions of a solid or multi-layer polymer medical device (e.g., catheter) could be loaded with magnetically susceptible particles or solids to generate artifacts at the tip of the medical device, at predefined bends along the elongate member of the medical device, and/or at locations where bends should be formed during use. In either of these alternative embodiments, magnetically susceptible particles or solids need not be compounded into the devices main structure (e.g., polymer) directly. Instead, magnetically susceptible particle filled or compounded polymers could be cast or molded into discrete marker structures separately and integrated into the device during manufacturing, as described herein. Alternatively, in embodiments in which a magnetically susceptible material is compounded into a base material (e.g. material that has a magnetic susceptibility that is less than the magnetic susceptibility of the material that forms the magnetically susceptible particles), the base material can be utilized in any suitable manufacturing process to form a medical device, such as modern 3D printing processes, fused deposition modeling, stereolithography additive printing, and/or selective laser sintering. Therefore, magnetically susceptible particles or solids can be compounded with a polymer and extruded into filaments, suspended in a photopolymer, or mixed with a base polymer in pulverized form for sintering. At least one part or feature of an interventional medical device could then be constructed from additive layers of any of these compounded polymers to make at least one part or feature of the device visible under MRI. For example, the magnetically susceptible suspension could be used to construct an entire medical device (e.g., solid or hollow shaft) or at least one select feature (e.g., covering, coating, jacket, marker bands or segments, molded components) of a medical device. Alternatively, the construct can be an extruded tube where co-extrusion or intermittent extrusion can be used to encapsulate an entire magnetically susceptible material (e.g., magnetically susceptible metallically doped substrate).

FIG. 25 illustrates an example alternative medical device 240 that can be included in a medical system. In this example, the medical device 240 is an alternative embodiment of a cannula 242 that can be included in a medical system. The medical device 240 includes a first portion 242 formed as an outer lubricious layer, a second portion 242 formed as an intermediate layer, and third portion 246 formed as an inner layer. The first portion 240 is formed of a first material, the second portion 242 is formed of a second material, and the third portion 246 is formed of a third material. The first material is different than the second and third materials and has a first magnetic susceptibility. The second material (e.g., suspension material, such as those described herein (e.g., magnetically susceptible particles (e.g., metallic particles) suspended in a material that is different than the material that forms the magnetically susceptible particles)) is different than the first and third materials and has a second magnetic susceptibility that is different than (e.g., greater than, less than) the first magnetic susceptibility. The third material is different than the first and second materials and has a third magnetic susceptibility that is different than (e.g., greater than, less than) the second magnetic susceptibility and different than, equal to, less than, or greater than the first magnetic susceptibility. For example, the first material can be a polymer, the second material can be PFTE that includes magnetically susceptible particles (e.g., stainless steel powder), and the third material can be a polymer.

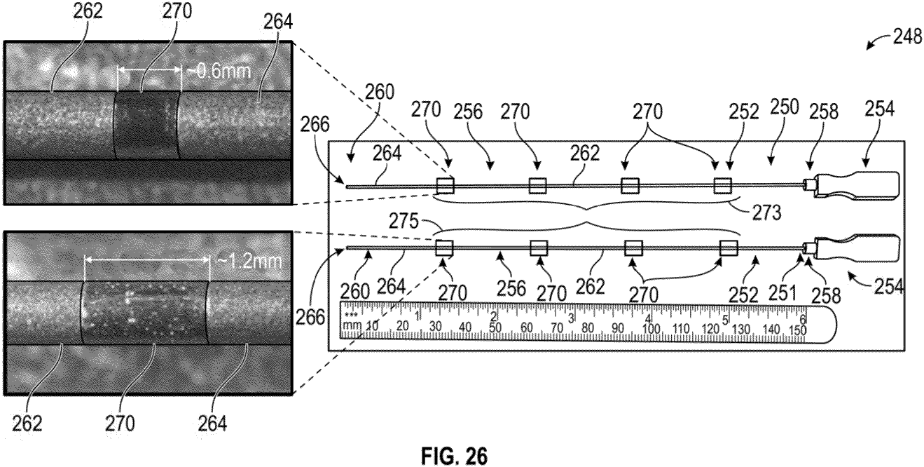

As described herein, visualization of devices during the performance of a procedure (e.g., treatment) is a fundamental challenge in interventional MRI. Conventional interventional devices and materials are either highly susceptible, resulting in artifacts that obliterate key features of the imaged tissue/field, or minimally susceptible, leaving devices not visible for identification or tracking within the image. Therefore, there is a need to leverage diagnostic imaging modalities, MR in particular, directly in guiding therapy. Identification and precise targeting of lesions or abnormal anatomy is a significant clinical challenge. The medical devices described herein are considered advantageous at least because they can substantially improve the ability of clinicians to precisely visualize, identify, track, and navigate medical devices to various locations within the body and improve overall patient care (e.g., diagnosis, treatment delivery) by locating a marker along the lengthwise axis of the medical device with great accuracy. The markers described herein can be used with a medical device to generate identifiable patterns on interventional MR images allowing for a high degree of control over the position, size, and intensity of susceptibility artifacts generated by interventional devices. FIG. 26 illustrates a set of example alternative medical devices 250, 251 that can be included in a medical system separately, or in in combination. In this example, each of the medical devices 250, 251 is an alternative embodiment of a stylet 252 that can be included in a medical system, such as those described herein. The stylet 252 is similar to the stylet 50 illustrated in FIGS. 1, 3, 5, and 6 and described above, except as detailed below. In the illustrated embodiment, each stylet 252 has a hub member 254 and an elongate member 256. The elongate member 256 has a proximal end 258, a distal end 260, an outer surface 262, and a main body 264 that defines a distal tip 266.

The elongate member 256 includes a plurality of markers 270 (e.g., four markers) disposed between the proximal end 258 and the distal end 260 of the elongate member 256. In the embodiment shown, each marker of the plurality of markers 270 is formed as a single layer of a material (e.g., metal, metal alloy) deposited on the outer surface 262 of the elongate member 256 to form a distinct marker visible under MRI. Each marker of the plurality of markers 270 is a marker band that is disposed around the entire circumference of the outer surface 262 of the elongate member 256. However, alternative embodiments can include a marker having any suitable structural arrangement, such as a marker that only partially extends around the circumference of an outer surface of an elongate member, and those described herein.

Material deposited on a medical device to form a marker can be deposited to form any suitable shape having any suitable size and be formed of any suitable material having the magnetic susceptibility described herein. For example, when a marker (e.g., marker band) is formed using material deposited on a portion of a medical device, the marker can have any suitable width along a lengthwise axis of a medical device (e.g., elongate member of a medical device), such as widths equal to, less than, greater than, or about 0.6 millimeters, 1.2 millimeters, or between about 0.6 millimeters and about 1.2 millimeters, and any suitable thickness measured along a cross-section of a medical device (e.g., elongate member of a medical device) that are equal to, less than, greater than, or about 0.0006 inches. Optionally, multiple layers of material can be used to create a marker (e.g., two or more layers of the same material, or different, materials). In the illustrated embodiment, the first medical device 250 includes markers that have a width equal to about 0.6 millimeters and the second medical device 251 includes markers that have a width equal to about 1.2 millimeters.

In the illustrated embodiment, each marker of the plurality of markers 270 is formed of nickel, having a thickness equal to about 0.0006 inches, plated on a portion of the elongate member 256 using a nickel-strike electroplating process to deposit thin layers of nickel over portions of the elongate member 256. Examples of materials considered suitable to deposit on a medical device to form a marker include nickel plating (e.g., bright nickel plating), the materials described herein and having the magnetic susceptibilities described herein, and any other material according to an embodiment.

Placing a material onto a medical device (e.g., plating) can be accomplished using any suitable technique or method, such as electroplating, sputtering, striking, vapor deposition, chemical vapor deposition, coating, chemical baths, evaporative techniques, and any other technique or method considered suitable for a particular embodiment. Optionally, a material can be placed on a medical device subsequent to masking the medical device such that the area where a marker is desired is left exposed. Examples of suitable types of masking include heat-shrink tubing, taping, spray or dip coatings, photoresists, and/or molds (e.g., silicone mold) that inhibit deposition onto the surface of a base material. For example, a heat-shrink tubing (e.g., fluoropolymer heat-shrink tubing) can be used to selectively mask off segments of a medical device (e.g., elongate member) and leave portions of the medical device exposed for plating (e.g., 0.06 millimeter portion, 1.2 millimeter portion). In another example, a reusable silicone mold could be used to hold and mask off portions of a medical device, or multiple devices at the same time. Subsequent to masking, the exposed portions of the medical device can optionally be cleaned and plated (e.g., using a Wood’s nickel strike process, which can be used to promote the adherence of successive layers of electroplated nickel). Another example includes applying a masking material (e.g., polymer) over an entire medical device, or a portion of a medical device, and subsequently creating distinct markers on the medical device (e.g., using laser ablation to create distinct markers). Any mold used to create a marker can be removed prior to device use in a clinical setting.

Alternative to masking portions of a medical device, thin layers of magnetically susceptible material can be applied to parts, or the entirety, of a medical device and excess material removed to form distinct markers (e.g., during post processing). Subtractive processes considered suitable to form markers in this manner include sanding, grinding, etching, and any other technique or method considered suitable for a particular embodiment.

These embodiments are considered advantageous at least because they are readably manufacturable, scalable, will not change a medical device’s mechanical properties (e.g., three-point bending, tensile strength, column strength), and have a minimal effect on a device’s surface profile. In addition, these embodiments are considered advantageous at least because deposited thin films of materials offer a high degree of control over marker size and shape and can be concealed using over plating to prevent flaking and improve biocompatibility.

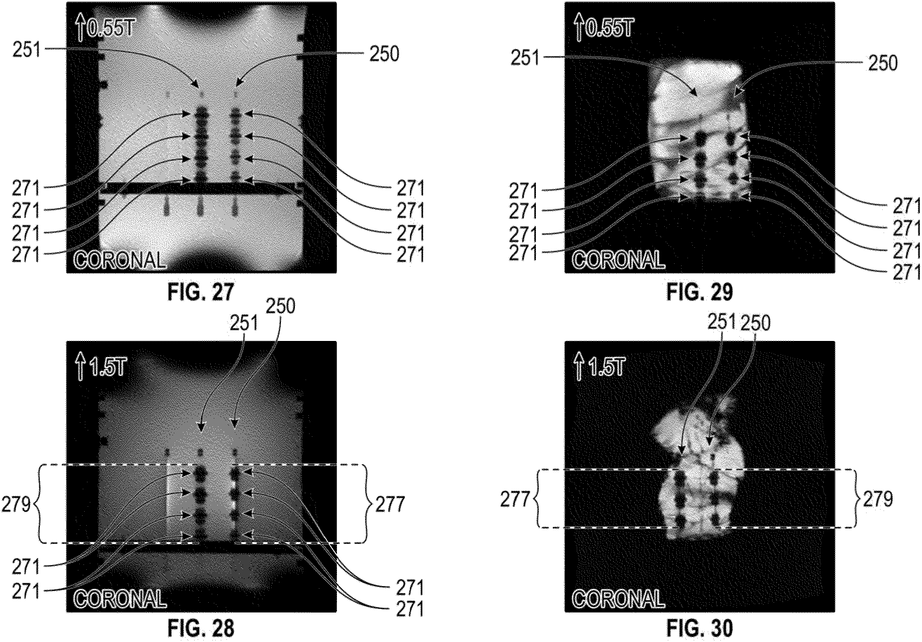

FIGS. 27 through 30 illustrate test results relating to the medical device 250 and the medical device 251. Both medical devices 250, 251 have been tested at 0.55 T, 1.5 T, and 3.0 T MRI field strengths in an imaging phantom material (e.g., copper sulfate solution) and porcine tissue. Any suitable type of imaging sequences can be used to view a marker under MRI, such as gradient refocusing echo, spin echo, true fast imaging with steady-state precession, fast low flip angle shot spoiled gradient-echo, and other functional imaging sequences.

As shown in FIGS. 27 through 30, the first medical device 250 includes a first pattern of markers 273 and the second medical device includes a second pattern of markers 275. The markers included on the first and second medical devices 250, 251 generate a repeatable and tunable image artifact 271 that can be used to visualize, track, and navigate a medical device during interventional MR procedures. In these procedures, image processing software or machine learning can optionally be used to determine the state of these markers in near real-time, aiding in the estimation of a devices positioning through automated sequence selection and allowing for path planning to targeted locations during therapy delivery in avoidance of critical or sensitive anatomy.

While the medical devices described herein have included cannulas and stylets, the markers described herein can be included on any suitable medical device and selection of a suitable medical device to include a marker can be based on various considerations including the intended use of the medical device on which a marker is disposed. Examples of medical devices considered suitable to include a marker include cannulas, needles, stylets, sheaths, catheters, balloon catheters, guidewires, introducers, implantable devices, and any other medical device considered suitable for a particular embodiment.

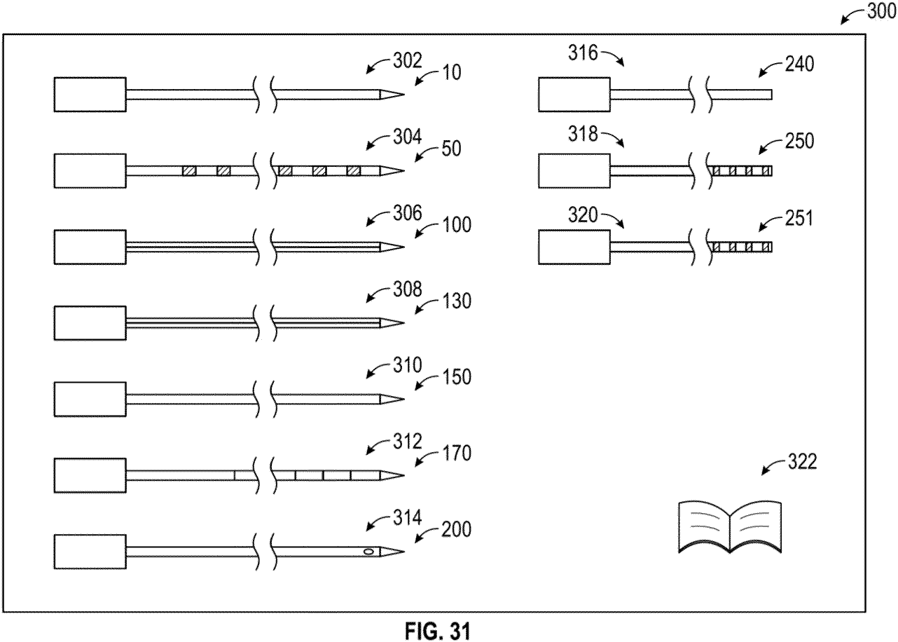

FIG. 31 illustrates an example kit 300 comprising a first medical device 302 according to an embodiment, such as the first medical device 10 illustrated in FIGS. 1 and 2; a second medical device 304 according to an embodiment, such as the second medical device 50 illustrated in FIGS. 1, 3, 5, and 6; a third medical device 306 according to an embodiment, such as the medical device 100 illustrated in FIG. 11; a fourth medical device 308 according to an embodiment, such as the medical device 130 illustrated in FIG. 13; a fifth medical device 310 according to an embodiment, such as the medical device 150 illustrated in FIG. 14; a sixth medical device 312 according to an embodiment, such as the medical device 170 illustrated in FIG. 15; a seventh medical device 314 according to an embodiment, such as the medical device 200 illustrated in FIGS. 16, 17, and 18; an eighth medical device 316 according to an embodiment, such as the medical device 240 illustrated in FIG. 25; a ninth medical device 318 according to an embodiment, such as the medical device 250 illustrated in FIG. 26; a tenth medical device 320 according to an embodiment, such as the medical device 251 illustrated in FIG. 26; and instructions for use 322.

While kit 300 has been illustrated as including ten medical devices 302, 304, 306, 308, 310, 312, 314, 316, 318, and 320, any suitable number, and type, of medical devices can be included in a kit. Selection of a suitable number of medical devices to include in a kit according to a particular embodiment can be based on various considerations, such as the treatment intended to be performed. Examples of numbers of medical devices considered suitable to include in a kit include at least one, one, two, a plurality, three, four, five, six, seven, eight, nine, ten, more than ten, and any other number considered suitable for a particular embodiment. In an alternative embodiment, a kit can include one or more medical systems, such as those described herein, and include any suitable number and type of medical devices, such as those described herein.

Furthermore, while medical device 10, medical device 50, medical device 100, medical device 130, medical device 150, medical device 170, medical device 200, medical device 240, medical device 250, and medical device 251 have been illustrated as included in kit 300, any suitable medical device can be included in a kit. Selection of a suitable medical device to include in a kit according to a particular embodiment can be based on various considerations, such as the treatment intended to be performed. Examples of medical devices considered suitable to include in a kit include those described herein, variations of those described herein, one or more inner sheaths, one or more outer sheaths, one or more access needles, and/or any other medical device considered suitable for a particular embodiment.

The inclusion of the medical devices described herein in a kit is considered advantageous at least because in some alternative kit embodiments a single needle (e.g., medical device 10) can be packaged with a plurality of secondary medical device (e.g., stylets) and the needle can be used with each of the secondary medical devices independently, or concurrently. Each of the secondary medical devices can be used under MRI using multiple MR field strengths, or can be specifically tailored to accommodate multiple MR-guided interventional procedures (e.g., treatment).

For example, in embodiments in which a kit includes a plurality of medical devices (e.g., a needle and a plurality of stylets), a first medical device (e.g., stylet) can include a material (e.g., marker) tailored for (e.g., visible under) a first MR field strength (e.g., 0.55 T), a second medical device (e.g., stylet) can include a material (e.g., marker) tailored for (e.g., visible under) a second MR field strength (e.g., 1.5 T), and a third medical device (e.g., stylet) can include a material (e.g., marker) tailored for (e.g., visible under) a third MR field strength (e.g., 3 T). The first MR field strength can be different, or the same as, the second MR field strength and/or the third MR field strength. The second MR field strength can be different, or the same as, the first MR field strength and/or the third MR field strength. The third MR field strength can be different, or the same as, the first MR field strength and/or the second MR field strength. This embodiment can be accomplished using materials (e.g., metals, metal alloys, the materials described herein) with targeted magnetic susceptibility, geometric sizes positioned along a portion of the medical device (e.g., the length of an elongate member of the medical device). For example, the first medical device can have a first type of marker and a first magnetic susceptibility, the second medical device can have a second type of marker and a second magnetic susceptibility, and the third medical device can have a third type of marker and a third magnetic susceptibility. The first type of marker can be the same as, or different than, the second type of marker and/or the third type of marker. The first magnetic susceptibility, or first susceptibility, can be the same as, or different than (e.g., greater than, less than), the second magnetic susceptibility, or second susceptibility, and/or the third magnetic susceptibility, or third susceptibility. The second type of marker can be the same as, or different than, the first type of marker and/or the third type of marker. The second magnetic susceptibility, or second susceptibility, can be the same as, or different than (e.g., greater than, less than), the first magnetic susceptibility, or first susceptibility, and/or the third magnetic susceptibility, or third susceptibility. The third type of marker can be the same as, or different than, the first type of marker and/or the second type of marker. The third magnetic susceptibility, or third susceptibility, can be the same as, or different than (e.g., greater than, less than), the first magnetic susceptibility, or first susceptibility, and/or the second magnetic susceptibility, or second susceptibility. Alternatively, medical devices (e.g., stylets), such as those described herein, can include markers covering a range of different interventional MRI procedures under a single or multiple field strengths can be included in a kit with a second MRI compatible medical device (e.g., needle). In a specific embodiment, a single titanium needle can be packaged with a plurality of stylets each of which can be used independently, or concurrently, depending on the MRI static field strength intended to be used or the specific interventional procedure intended to be completed. Alternatively, a marker included on a medical device could be tuned to a specific MR sequence.

In an example, a kit useful in performing treatment under magnetic resonance includes a first medical device of an embodiment, a second medical device of an embodiment, and a third medical device of an embodiment. The first medical device includes a first marker that produces a first image artifact under a first MR field strength. The second medical device includes a second marker that produces a second image artifact under a second MR field strength. The third medical device includes a third marker that produces a third image artifact under a third MR field strength. The first MR field strength is different than the second MR field strength and the third MR field strength. The second MR field strength is different than the third MR field strength. The first image artifact has a first set of characteristics (e.g., size, shape, pattern, and/or intensity) under the first MR field strength, a second set of characteristics under the second MR field strength, and a third set of characteristics under the third MR field strength. The first set of characteristics of the first image artifact being different than the second set of characteristics of the first image artifact and the third set of characteristics of the first image artifact. The second set of characteristics of the first image artifact being different than the third set of characteristics of the first image artifact. The second image artifact has a first set of characteristics under the first MR field strength, a second set of characteristics under the second MR field strength, and a third set of characteristics under the third MR field strength. The first set of characteristics of the second image artifact being different than the second set of characteristics of the second image artifact and the third set of characteristics of the second image artifact. The second set of characteristics of the second image artifact being different than the third set of characteristics of the second image artifact. The third image artifact has a first set of characteristics under the first MR field strength, a second set of characteristics under the second MR field strength, and a third set of characteristics under the third MR field strength. The first set of characteristics of the third image artifact being different than the second set of characteristics of the third image artifact and the third set of characteristics of the third image artifact. The second set of characteristics of the third image artifact being different than the third set of characteristics of the third image artifact.

The kits described herein advantageously provide a clinician with a single product (e.g., kit) that can be used under multiple settings. A titanium needle with a low magnetic susceptibility allows for positioning precision near the tip where treatment is being performed (e.g., a biopsy is being taken), meanwhile markers on one or more removable stylets allow for real-time trackability at all field strength or across multiple interventional procedures. While this example embodiment describes use of a needle and a plurality of stylets, such an embodiment can include any suitable combination of medical devices, such as those described herein (e.g., sheaths, removable dilators). For example, a sheath that has a low magnetic susceptibility (e.g., creates a small image artifact under MRI) can be packaged with a plurality of removable dilators, each of which include one or more markers (e.g., different magnetic susceptibility, high relative magnetic susceptibility creating a large image artifact under MRI) to facilitate use under various MRI static field strengths.

Various methods of performing interventional medical treatment under MRI are described herein. While the methods described herein are shown and described as a series of acts, it is to be understood and appreciated that the methods are not limited by the order of acts, as some acts may in accordance with these methods may be omitted, occur in the order shown and/or described, occur in different orders, and/or occur prior to, subsequent to, or concurrently with other acts described herein.

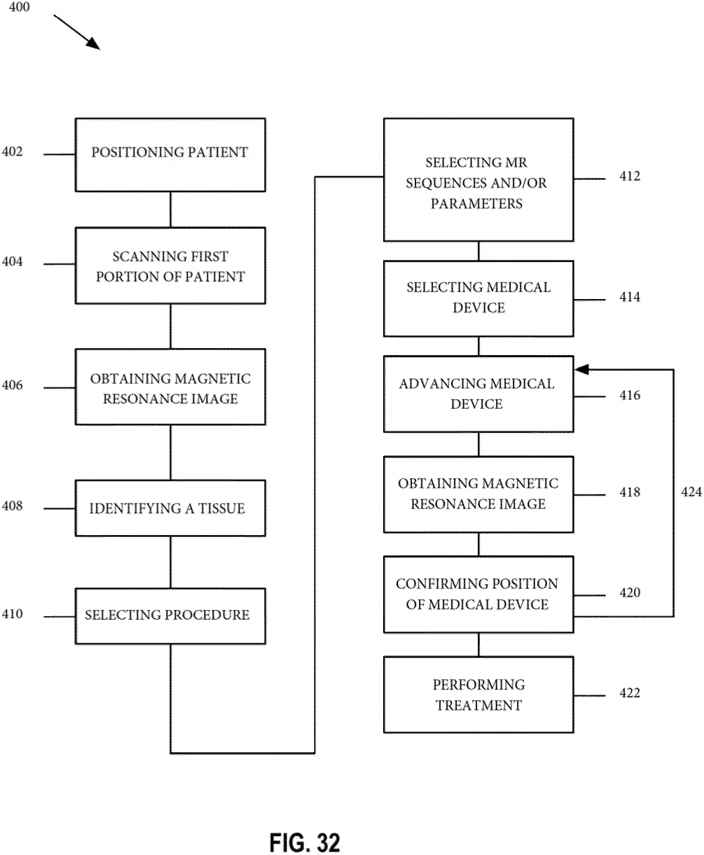

FIG. 32 illustrates a schematic illustration of an example method 400 of performing treatment under MRI.

An initial step 402 comprises positioning a patient within a MR scanner. Another step 404 comprises scanning a first portion of the patient using the MR scanner. Another step 406 comprises obtaining a MR image of the first portion of the patient. Another step 408 comprises identifying a tissue that has predefined characteristics using the MR image. Another step 410 comprises selecting a procedure to treat the tissue based upon the predefined characteristics. Another step 412 comprises selecting MR sequences and/or parameters to use during performance of the selected procedure. Another step 414 comprises selecting a medical device to accomplish performance of a medical procedure based upon the selected MR sequences and/or parameters intended to be used during performance of the selected procedure. While the patient remains positioned within the MR scanner used to scan a portion of the patient, another step 416 comprises advancing the medical device into a bodily passage and to the tissue while scanning a second portion of the patient that includes the medical device using the MR scanner. Another step 418 comprises obtaining a MR image of the second portion of the patient that includes the medical device. Another step 420 comprises confirming the position of the medical device within the bodily passage. Another step 422 comprises performing treatment using the medical device.

Step 402 can be accomplished by positioning a patient within any suitable MR scanner, having any suitable field strength, such as conventional MR scanners, MR scanners that can accomplish gradient refocusing echo imaging, MR scanners that can accomplish spin echo imaging, MR scanners that can accomplish true fast imaging with steady-state precession, MR scanners that can accomplish fast low flip angle shot spoiled gradient-echo imaging, MR scanners that utilize 0.55 T fields, 1.5 T fields, 3 T fields, fields between about 0.055 T and 1.5 T, fields less than 1 T, and any other MR scanner considered suitable for a particular embodiment.

Step 404 can be accomplished by scanning any suitable portion of a patient and selection of a suitable portion of a patient to scan can be based on various considerations, including the treatment intended to be performed. Examples of portions of a patient considered suitable to scan include the extremities (e.g., arms, legs), chest, breast, spine, neck, head, abdomen, pelvis, prostate, peri-prostatic structures, tissues surrounding the portions of a patient described herein, and/or any other portion of the patient considered suitable for a particular embodiment.

Step 406 can be accomplished by obtaining the MR image from the magnetic resonance scanner used in step 402.

Step 408 can be accomplished by reviewing the MR image obtained in step 406 and utilizing conventional techniques and/or methods to determine whether tissue has predefined characteristics (e.g., tissue has characteristics indicative of cancer, is a lesion, abnormal mass). Furthermore, the margins of any tissue (e.g., abnormal mass) can be identified and used in further steps, as described herein, to remove and/or treat the tissue.

Step 410 can be accomplished by reviewing the predefined characteristics identified in step 408 and selecting a procedure to treat the tissue based upon the predefined characteristics.

Step 412 can be accomplished based upon the procedure selected in step 410, the available MR scanner, and/or utilizing any suitable number and/or type of sequence and/or parameter. An MR scanner can have any suitable number and type of MR image parameters, such as gradient refocusing echo imaging, spin echo imaging, true fast imaging with steady-state precession, fast low flip angle shot spoiled gradient-echo imaging, field strengths, such as 0.55 T, 1.5 T, 3 T, between about 0.055 T and 1.5 T, and fields less than 1 T, slice thickness, flip angle, field-of-view, resolution, gradient fields, and any other image parameter considered suitable for a particular embodiment. An MR scanner can have any suitable number and type of MRI sequence parameters (SE/GRE, TR, TE, flip angle, field-of-view, resolution, slice thickness, gradient field). Optionally, MR sequences and/or parameters can be modified throughout performance of a procedure to accommodate a medical system and/or medical device.

Step 414 can be accomplished using one or more of the medical devices described herein, a medical system described herein, or any other suitable medical device or medical system. Alternatively, a medical device or medical system can be selected from a kit, such as kit 300, and selection of a medical device or medical system can be based upon the MR field selected in step 412.

Step 416 can be accomplished using the medical device selected in step 414. Alternatively, step 416 can be accomplished using any suitable medical device, or medical system, such as the medical devices incorporated by reference herein. Examples of medical devices considered suitable to use to complete a method of treatment and of other steps considered suitable to include in a method of treatment are described in U.S. Pat. Application No.: 17/573,087, filed on Jan. 11, 2022, which is hereby incorporated by reference in its entirety for the purpose of describing medical devices considered suitable to complete a method of treatment and for the purpose of describing steps considered suitable to include in a method of treatment. Step 416 can be accomplished by applying a distally-directed force on the medical device such that a distal end of the medical device is advanced into a bodily passage and to, within, or adjacent to, the tissue that has been identified as having the predefined characteristics. Alternatively, in embodiments in which a medical system is being used to accomplish step 416, step 416 can be accomplished in two steps, which can be accomplished concurrently, or separately. A first step comprises applying a distally-directed force on a first medical device of a medical system such that a distal end of the first medical device is advanced into a bodily passage and to, within, or adjacent to, the tissue that has been identified as having the predefined characteristics. A second step comprises applying a distally-directed force on a second medical device of a medical system such that a distal end of the second medical device is advanced into a lumen defined by the first medical device and advanced to, within, or adjacent to, the tissue that has been identified as having the predefined characteristics.

A bodily passage can include any suitable portion of a body, including existing bodily passages, bodily lumens, and/or bodily passages created through tissues layers and/or fascia using a device described herein. Step 416 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the medical device, portions that include the tissue that has predefined characteristics and the medical device, and any other portion of the patient considered suitable for a particular embodiment. For example, a second portion of the patient can be the same as, or different than, the first portion of the patient.

Step 418 can be accomplished by obtaining the MR image from the MR scanner used in step 402.

Step 420 can be accomplished by reviewing the MR image obtained in step 418 and confirming the medical device is positioned at a desired location within the bodily passage (e.g., at, within, or adjacent to, the tissue that has been identified as having the predefined characteristics). This can be accomplished by visualizing one or more markers, or an image artifact, or plurality of image artifacts, created by the one or more markers, included on the medical device, as described herein. If the medical device is not positioned at a desired location, an optional step 424 comprises repeating step 416, step 418, and step 420 to manipulate the position of the medical device (e.g., in real time, while scanning a portion of the patient that includes the tissue and/or medical device). While step 416, step 418, and step 420 have been illustrated as separate steps, these steps can optionally be completed simultaneously in real time (e.g., while scanning a portion of the patient that includes the tissue and/or medical device). Optional step 424 can be accomplished any suitable number of times to correctly position a medical device. Alternatively, in embodiments in which a medical system is being utilized, step 420 can be accomplished by reviewing the MR image obtained in step 418 and confirming the medical system (e.g., the first medical device and/or second medical device) is positioned at a desired location within the bodily passage (e.g., at, within, or adjacent to, the tissue that has been identified as having the predefined characteristics).

Step 422 can be accomplished by performing treatment using the medical device. Any suitable treatment can be performed and based upon the tissue identified in step 408. Examples of treatments considered suitable to perform include treatments utilizing the medical device, biopsies, and any other treatment considered suitable for a particular embodiment. Alternatively, in embodiments in which a medical system is being utilized, step 422 can be performed using the first medical device and/or the second medical device.

Each of step 404, step 406, step 408, step 410, step 412, step 414, step 416, step 418, step 420, step and/or step 422 can be accomplished without removing the patient from the MR scanner within which the patient is positioned in step 402. In an alternative embodiment, however, a patient can be moved (e.g., outside of the MR scanner within which the patient is positioned in step 402, to a different location) between one or more steps described herein, if desired.

Method 400 is considered advantageous at least because each step of method 400 can be performed during a single patient (e.g., same day) visit and using the same MR scanner, which increases efficiency and reduces the number of patient visits and procedures performed. This results in a set of procedures in which a physician can visualize, diagnose, and treat a patient in a single patient visit. While some steps have been described as being completed while scanning a portion of the patient using a MR scanner and other steps have not been described as being performed while scanning a portion of the patient using a MR scanner, any step described herein can be completed while scanning a portion of a patient using the MR scanner, and/or an ultrasound device or without scanning a portion of a patient using a MR scanner. In embodiments in which an ultrasound image is obtained, the MR image obtained can be electronically fused with a real-time ultrasound image (e.g., transrectal ultrasound image of a prostate). While some steps have been described as being completed while scanning a portion of the patient using a MR scanner, this step can be broken into two separate steps such that a subsequent step of scanning a portion of a patient using the MR scanner can be accomplished. Furthermore, any step which is completed while scanning a portion of the patient using the MR scanner can comprise obtaining a single still image and be repeated any desired number of times to obtain multiple MR images that can be grouped as a cine to show motion and/or any step which is completed while scanning a portion of the patient using the MR scanner can comprise obtaining a live image, such as being completed under live real-time MRI visualization.

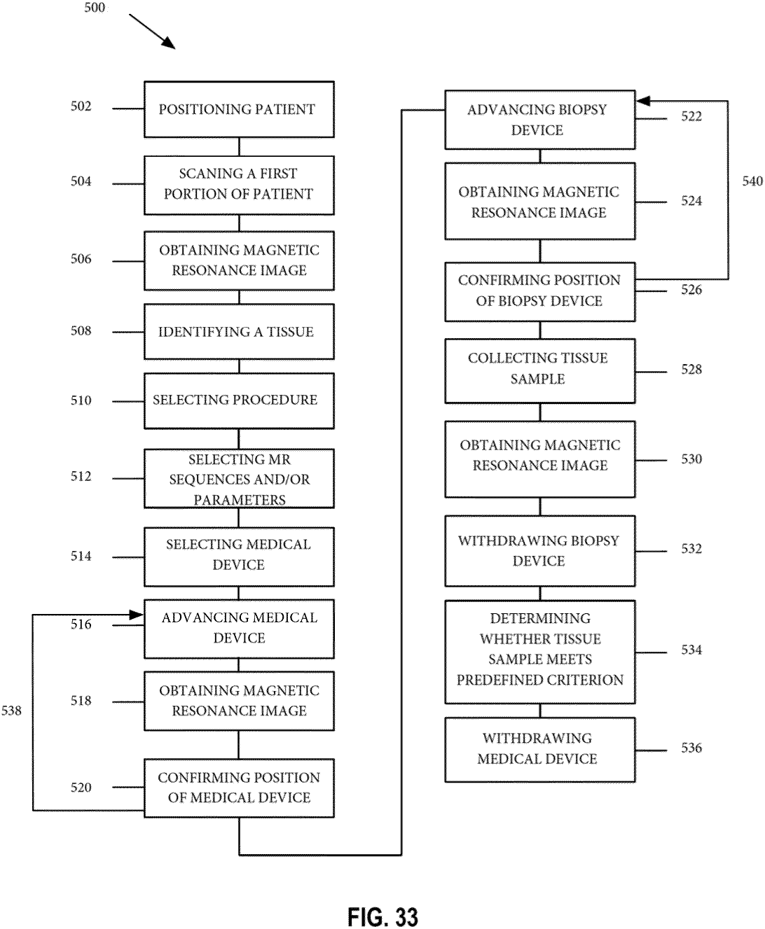

FIG. 33 illustrates a schematic illustration of another example method 500 of performing treatment under MRI.

An initial step 502 comprises positioning a patient within a MR scanner. Another step 504 comprises scanning a first portion of the patient using the MR scanner. Another step 506 comprises obtaining a MR image of the first portion of the patient. Another step 508 comprises identifying a tissue that has predefined characteristics using the MR image. Another step 510 comprises selecting a procedure to treat the tissue based upon the predefined characteristics. Another step 512 comprises selecting MR sequences and/or parameters to use during performance of the selected procedure. Another step 514 comprises selecting a medical device to accomplish performance of a medical procedure based upon the selected MR sequences and/or parameters intended to be used during performance of the selected procedure. While the patient remains positioned within the MR scanner used to scan a portion of the patient, another step 516 comprises advancing a medical device into a bodily passage and to the tissue while scanning a second portion of the patient that includes the medical device using the MR scanner. Another step 518 comprises obtaining a MR image of the second portion of the patient that includes the medical device. Another step 520 comprises confirming the position of the medical device within the bodily passage. Another step 522 comprises advancing a biopsy device through the medical device and to the tissue while scanning a third portion of the patient that includes the biopsy device using the MR scanner. Another step 524 comprises obtaining a MR image of the third portion of the patient that includes the biopsy device. Another step 526 comprises confirming the position of the biopsy device. Another step 528 comprises collecting a tissue sample from the tissue using the biopsy device while scanning a fourth portion of the patient that includes the biopsy device and the tissue using the MR scanner. Another step 530 comprises obtaining a MR image of the fourth portion of the patient that includes the biopsy device. Another step 532 comprises withdrawing the biopsy device and the tissue sample through the medical device. Another step 534 comprises determining whether the tissue sample meets a predefined criterion. Another step 536 comprises withdrawing the medical device.

Step 502 can be completed as described herein with respect to step 402. Step 504 can be completed as described herein with respect to step 404. Step 506 can be completed as described herein with respect to step 406. Step 508 can be completed as described herein with respect to step 408. Step 510 can be completed as described herein with respect to step 410. Step 512 can be completed as described herein with respect to step 412. Step 514 can be completed as described herein with respect to step 414. Step 516 can be completed as described herein with respect to step 416. Step 518 can be completed as described herein with respect to step 418. Step 520 can be completed as described herein with respect to step 420.

If the medical device is not positioned at a desired location, an optional step 538 comprises repeating step 516, step 518, and step 520 to manipulate the position of the medical device (e.g., in real time, while scanning a portion of the patient that includes the tissue and/or medical device). While step 516, step 518, and step 520 have been illustrated as separate steps, these steps can optionally be completed simultaneously in real time (e.g., while scanning a portion of the patient that includes the tissue and/or medical device). Optional step 538 can be accomplished any suitable number of times to correctly position a medical device.

Step 522 can be accomplished by applying a distally-directed force on the biopsy device such that a distal end of the biopsy device is advanced into a lumen defined by the medical device, through the lumen defined by the medical device, and to the tissue that has been identified as having the predefined characteristics. Step 522 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the biopsy device, portions that include the tissue that has predefined characteristics and the biopsy device, and any other portion of the patient considered suitable for a particular embodiment. For example, a third portion of the patient can be the same as, or different than, the first portion of the patient and/or the second portion of the patient.

Step 522, step 524, step 526, step 528, step 530, and/or step 532 can be accomplished using any suitable biopsy device, such as MRI compatible biopsy devices, devices under the trade name MReye® provided by Cook Medical, the Echotip ProCore provided by Cook Medical, the Echotip Ultra provided by Cook Medical, and any other biopsy device considered suitable for a particular embodiment. Alternatively, step 522, step 524, step 526, step 528, step 530, and/or step 532 can be completed using a biopsy device that is not MRI compatible. This alternative step, or alternative steps, can comprise advancing the biopsy device through the medical device and to the tissue without scanning a portion of the patient that includes the biopsy device using the magnetic resonance scanner. An alternative to step 528 can comprise collecting a tissue sample using the biopsy device without scanning a portion of the patient that includes the biopsy device and the tissue using the magnetic resonance scanner.

Step 524 can be accomplished by obtaining the MR image from the magnetic resonance scanner used in step 402.

Step 526 can be accomplished by reviewing the MR image obtained in step 524 and confirming the biopsy device is positioned at a desired location within the bodily passage (.e.g., at, within, or adjacent to, the tissue that has been identified as having the predefined characteristics). This can be accomplished, for example, by including one or more markers, as described herein, on a distal portion of the biopsy device. If the biopsy device is not positioned at a desired location, an optional step 540 comprises repeating step 522, step 524, and step 526 to manipulate the position of the biopsy device (e.g., in real time, while scanning a portion of the patient that includes the tissue and/or medical device). While step 522, step 524, and step 526 have been illustrated as separate steps, these steps can optionally be completed simultaneously in real time (e.g., while scanning a portion of the patient that includes the tissue and/or medical device). Optional step 540 can be accomplished any suitable number of times to correctly position a biopsy device and be utilized during the performance of a procedure any time a biopsy device, medical system, and/or device is advanced, an MR image is obtained, and/or confirmation of the position of the medical system and/or device is completed.

Step 528 can be accomplished using the biopsy device and conventional methods of obtaining a sample using a biopsy device. Step 528 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include the biopsy device, portions that include the tissue that has predefined characteristics and the biopsy device, and any other portion of the patient considered suitable for a particular embodiment. For example, a fourth portion of the patient can be the same as, or different than, the first portion of the patient, the second portion of the patient, and/or the third portion of the patient.

Step 516, step 522, and/or step 528 can optionally be conducted in combination with performing an ultrasound on the portion of the patient that includes a medical device and/or biopsy device. In embodiments in which an ultrasound image is obtained, the MR image obtained can be electronically fused with a real-time ultrasound image (e.g., transrectal ultrasound image of a prostate).

Step 530 can be accomplished by obtaining the MR image from the MR scanner used in step 402. An optional step comprises confirming the tissue sample has been collected. This optional step can be accomplished by reviewing the MR image obtained in step 530 and confirming the biopsy device has collected the tissue sample (e.g., the tissue that has been identified as having the predefined characteristics). If the biopsy device has not collected the tissue sample, optional steps comprise repeating step 522, step 524, step 526, step 528, step 530, and/or this optional step.

Step 506, step 518, step 524, and/or step 530 can comprise obtaining a single still image. Alternatively, step 504, step 516, step 522, and/or step 528 can be repeated any desired number of times such that step 506, step 518, step 524, and/or step 530 comprises obtaining multiple magnetic resonance images of a portion that can be grouped as a cine to show motion.

Step 532 can be accomplished by applying a proximally-directed force on the biopsy device such that it is withdrawn from the lumen defined by the medical device. In an alternative embodiment, step 522, step 524, step 526, step 528, step 530, and step 532, can be omitted from method 500, and other methods described herein, and the medical device advanced in step 516 can be utilized to obtain a biopsy and steps similar to those described with respect to a biopsy device can be completed utilizing the medical device.

Step 534 can be accomplished using any technique or method considered suitable to determine whether tissue meets predefined criterion. For example, step 534 can utilize conventional techniques and methods for determining whether a tissue sample is malignant, such as frozen section and/or other cytological methods.

Each of step 504, step 506, step 508, step 510, step 512, step 514, step 516, step 518, step 520, step 522, step 524, step 526, step 528, step 530, step 532, step 534, and/or step 536 can be accomplished without removing the patient from the magnetic resonance scanner within which the patient is positioned in step 502. In an alternative embodiment, however, a patient can be moved (e.g., outside of the MR scanner within which the patient is positioned in step 502, to a different location) between one or more steps described herein, if desired.

Method 500 is considered advantageous at least because each step of method 500 can be performed during a single patient visit (e.g., same day) and using the same MR scanner, which increases efficiency and reduces the number of patient visits and procedures performed. This results in a set of procedures in which a physician can visualize, diagnose, and treat a patient in a single patient visit. While some steps have been described as being completed while scanning a portion of the patient using a MR scanner and other steps have not been described as being performed while scanning a portion of the patient using a MR scanner, any step described herein can be completed while scanning a portion of a patient using the MR scanner, and/or an ultrasound device or without scanning a portion of a patient using a MR scanner. In embodiments in which an ultrasound image is obtained, the MR image obtained can be electronically fused with a real-time ultrasound image (e.g., transrectal ultrasound image of a prostate). While some steps have been described as being completed while scanning a portion of the patient using a MR scanner, this step can be broken into two separate steps such that a subsequent step of scanning a portion of a patient using the magnetic resonance scanner can be accomplished. Furthermore, any step which is completed while scanning a portion of the patient using the MR scanner can comprise obtaining a single still image and be repeated any desired number of times to obtain multiple MR images that can be grouped as a cine to show motion and/or any step which is completed while scanning a portion of the patient using the MR scanner can comprise obtaining a live image, such as being completed under live real-time MRI visualization.

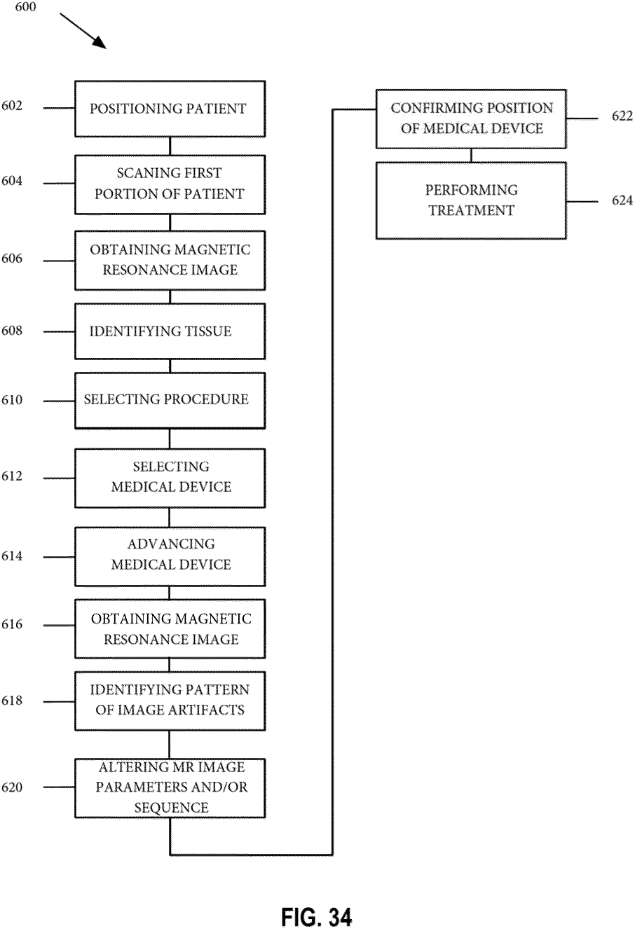

FIG. 34 illustrates a schematic illustration of an example method 600 of performing treatment under MRI.

An initial step 602 comprises positioning a patient within a MR scanner. Another step 604 comprises scanning a first portion of the patient using the MR scanner. Another step 606 comprises obtaining a MR image of the first portion of the patient. Another step 608 comprises identifying a tissue that has predefined characteristics using the MR image. Another step 610 comprises selecting a procedure to treat the tissue based upon the predefined characteristics. Another step 612 comprises selecting a medical device that has a first pattern of markers to accomplish performance of the procedure. While the patient remains positioned within the MR scanner used to scan the first portion of the patient, another step 614 comprises advancing the medical device that has the first pattern of markers into a bodily passage and to the tissue while scanning a second portion of the patient that includes the medical device and a first pattern of image artifacts produced by the first pattern of markers using the MR scanner. Another step 616 comprises obtaining a MR image of the second portion of the patient that includes the medical device and the first pattern of image artifacts produced by the first pattern of markers. Another step 618 comprises identifying the first pattern of image artifacts within the MR image. Another step 620 comprises altering a first set of MR image parameters and/or sequence to a second set of MR image parameters and/or sequence based upon the identified first pattern of image artifacts. Another step 622 comprises confirming the position of the medical device within the bodily passage. Another step 624 comprises performing treatment.

Step 602 can be completed as described herein with respect to step 402. Step 604 can be completed as described herein with respect to step 404. Step 606 can be completed as described herein with respect to step 406. Step 608 can be completed as described herein with respect to step 408. Step 610 can be completed as described herein with respect to step 410.

Step 612 can be accomplished using one or more of the medical devices described herein, one or more of the medical devices included in the medical device systems described herein, and/or one or more of the medical devices included in the kits described herein. Each of the medical devices described herein includes a pattern of markers. For example, FIG. 26 illustrates a first medical device 250 having a first pattern of markers 273 and a second medical device having a second pattern of markers 275. Each marker of the plurality of markers 270 included in the first pattern of markers 273 and the second pattern of markers 275 has a magnetic susceptibility that produces an image artifact 271, as shown in FIGS. 27 through 30. Alternatively, step 612 can comprise selecting a first medical device and a second medical device that combined have a first pattern of markers.

Step 614 can be accomplished by applying a distally-directed force on the medical device such that a distal end of the medical device is advanced into a bodily passage and to, within, or adjacent to, the tissue that has been identified as having the predefined characteristics. Step 614 can be accomplished by scanning any suitable portion of a patient and selection of a suitable portion of a patient to scan can be based on various considerations, including the location of the tissue that has predefined characteristics. Examples of portions of a patient considered suitable to scan include portions that include the tissue that has predefined characteristics, portions that include a medical device that has a first pattern of markers producing a first pattern of image artifacts, portions that include the tissue that has predefined characteristics and a medical device that has a first pattern of markers producing a first pattern of image artifacts, portions that include a first medical device and a second medical device that combined have a first pattern of markers producing a first pattern of image artifacts, portions that include the tissue that has predefined characteristics and a first medical device and a second medical device that combined have a first pattern of markers producing a first pattern of image artifacts, and any other portion of the patient considered suitable for a particular embodiment. For example, a second portion of the patient can be the same as, or different than, the first portion of the patient.

Step 616 can be accomplished by obtaining the MR image from the MR scanner used in step 602 and step 616. FIGS. 29 and 30 illustrate an MR image showing example medical devices 250, 251 disposed within porcine tissue. The first medical device 250 produces a first pattern of image artifacts 277 and the second medical device 251 produces a second pattern of image artifacts 279. A medical device, or combination of a first medical device and a second medical device, can include any suitable pattern of markers to produce a pattern of image artifacts under MR imaging. For example, each medical device used to perform treatment, as described herein, or combinations thereof, can include a unique pattern of markers relative to the other medical devices used to perform treatment.

Step 618 can be accomplished by reviewing the MR image obtained in step 616 and identifying the first pattern of image artifact in the image. This can be accomplished using software (e.g., software with artifact recognition, localization, tracking, image processing, and/or machine learning) included in the MR scanner used in step 602 and/or visually by a clinician. For example, the MR scanner used in step 602, and the remainder of steps included in method 600, includes a database that correlates a unique pattern of image artifacts to each medical device, or combination of medical devices, used to perform treatment such that when a pattern of image artifacts is identified by the MR scanner during use the MR scanner can identify and/or track the medical device, or combination of medical devices, alter a first set of MR image parameters and/or sequence to a second set of MR image parameters and/or sequence, as described herein, and/or alter the image created by the MR scanner to create an altered MR image. The database can include features of each pattern of image artifacts produced by a medical device, or combination of medical devices, such as characteristics, such as size, shape, pattern, and/or intensity to identify specific medical devices. For example, each of the medical devices 50, 100, 130, 150, 170, 200, 240, 250, 251 has a distinct pattern of markers relative to the other medical devices. As a result, each of the medical devices 50, 100, 130, 150, 170, 200, 240, 250, 251, or combination of medical devices, will produce a distinct image artifact (e.g., code) under MRI that can be identified using the database included in the MR scanner. Image processing software or machine learning can optionally be used to determine the state of these markers, or image artifacts, in near real-time, aiding in the estimation of device positioning through automated sequence selection and allowing for path planning to targeted locations during treatment to avoid critical or sensitive anatomy.

Step 620 can be accomplished using software included in the MR scanner used in step 602 and/or a clinician. This step can be accomplished any time a MR image is taken and/or an image artifact is detected such that the MR image parameters and/or sequence are regularly altered or updated based upon image artifacts produced by a marker, or a plurality of markers, disposed on a first medical device, a second medical device, and/or a combination of first and second medical devices. An altered MR image can include a superimposed graphic of the medical device (e.g., portion of medical device, entirety of medical device), or combination of medical devices, identified using the first pattern of image artifacts, can include an amplified, attenuated, or omitted portion of an identified medical device (e.g., distal end), combination of medical devices, image artifact, or other object within the image, and/or include any other image manipulation considered suitable for a particular embodiment.

Interventional MR procedures generally require frequent adjustments or changes to MR imaging sequence parameters throughout various stages of a given procedure. For example, a vascular procedure may be best performed with one sequence type during device tracking (e.g., rapid frame rate, lower resolution, enhancement of certain tissue characteristics) and another sequence type once the interventional devices have been advanced to the target site (e.g., higher resolution, enhancement of different tissue characteristics to detect lesions, such as inflammation, ADC). Alternatively, one or more interventional devices may be employed across multiple MRI-based procedures, requiring specific imaging sequences to the particular device and procedure configuration. For instance, an MRI-compatible needle may be used across multiple soft-tissue biopsy procedures, each requiring different gross or fine tracking characteristics and highlighting different anatomy or tissue types. Currently, many of these imaging protocols involve manual optimization by the clinician using the MR scanner, which can introduce intra-operator variability and inefficiency.

Therefore, method 600 provides a mechanism for at least increasing the efficiency of performing treatment. For example, the software included on an MR scanner, such as those utilized to complete the methods described herein, can be utilized to provide automation of parameter adjustments throughout a procedure, or across multiple procedures, based upon identified patterns of image artifacts produced by a pattern of markers provided on a medical device, based upon identified patterns of image artifacts produced by a pattern of markers provided on a second medical device, and/or based upon identified patterns of image artifacts produced by a pattern of markers provided on first and second medical devices, as described herein. Therefore, the existence of an image artifact, or artifacts, can be used to automate the selection of MRI sequence parameters.

Method 600 can be used to automatically alter imaging parameters and/or sequence within a procedure, or across multiple procedures, using the same medical device, or combination of medical devices. For instance, changes in MRI sequence parameters (SE/GRE, TR, TE, flip angle, field-of-view, resolution, slice thickness, gradient field) can be triggered by the identification of an image artifact by the MR scanner. These image artifact driven triggers could be used to signal transitions in device tracking, guiding how MR sequences are adapted to follow a device or component amongst a field of multiple devices, and/or tailor an MR sequence to highlight characteristic of a particular tissue during a specific part of a procedure.

For example, a series of sequential MRI sequence parameters could be triggered successively based on an identified image artifact. This process could then be used to automate imaging protocols throughout an entire procedure. Alternatively, utilizing a first medical device and one or more second medical devices that is/are different than, but compatible with the first medical device (e.g., various guiding or balloon catheters), each with one or more pattern of markers, the specific imaging procedure relating to the use of the one or more second medical devices and the first medical device could be automated based on the identification of an image artifact generated by the combination of the first medical device and the one or more second medical devices. Using the detection of this pairing, a preprogrammed set of MRI sequences could be automatically loaded and triggered by identification of the combined image artifacts. In other words, a combination of image artifacts generated by the combination of susceptibility markers on two or more medical devices would serve as a unique pattern of image artifacts to identify a particular medical device pairing or device configuration that the MR scanner uses to automatically reconfigure a parameter, sequence, procedure, or the next step in the sequence or procedure.

Step 620 can be completed as described herein with respect to step 420. Step 622 can be completed as described herein with respect to step 422.

Each of step 604, step 606, step 608, step 610, step 612, step 614, step 616, step 618, step 620, and/or step 622 can be accomplished without removing the patient from the magnetic resonance scanner within which the patient is positioned in step 602. In an alternative embodiment, however, a patient can be moved (e.g., outside of the MR scanner within which the patient is positioned in step 602, to a different location) between one or more steps described herein, if desired.

Method 600 is considered advantageous at least because each step of method 600 can be performed during a single patient visit (e.g., same day) and using the same MR scanner, which increases efficiency and reduces the number of patient visits and procedures performed. This results in a set of procedures in which a physician can visualize, diagnose, and treat a patient in a single patient visit. While some steps have been described as being completed while scanning a portion of the patient using a MR scanner and other steps have not been described as being performed while scanning a portion of the patient using a MR scanner, any step described herein can be completed while scanning a portion of a patient using the MR scanner, and/or an ultrasound device or without scanning a portion of a patient using a MR scanner. In embodiments in which an ultrasound image is obtained, the MR image obtained can be electronically fused with a real-time ultrasound image (e.g., transrectal ultrasound image of a prostate). While some steps have been described as being completed while scanning a portion of the patient using a MR scanner, this step can be broken into two separate steps such that a subsequent step of scanning a portion of a patient using the magnetic resonance scanner can be accomplished. Furthermore, any step which is completed while scanning a portion of the patient using the MR scanner can comprise obtaining a single still image and be repeated any desired number of times to obtain multiple MR images that can be grouped as a cine to show motion and/or any step which is completed while scanning a portion of the patient using the MR scanner can comprise obtaining a live image, such as being completed under live real-time MRI visualization.

While a number of methods have been described herein, it will be appreciated that the method may be a non-invasive method that does not require an invasive intervention by a medical professional. For example, a method may be carried out within a body lumen or passageway, such as the ear canal or a nasal passage, in order to place a device within such a body lumen or passageway. Equally, methods may be implemented on a cadaver or artificial body parts, for example, for training purposes. Moreover, the skilled person will appreciate that the methods described herein may not be used on the human or animal body at all, but may be used in order to view other types of devices using MRI techniques, for example in an industrial setting.

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular arrangement of elements and steps disclosed herein have been selected by the inventor(s) simply to describe and illustrate examples of the invention and are not intended to limit the scope of the invention or its protection, which is to be given the full breadth of the appended claims and any and all equivalents thereof.

What is claimed is:

1. A medical system useful in performing treatment under magnetic resonance imaging comprising:
a first medical device formed of a first material, the first material having a first magnetic susceptibility;
a second medical device formed of a second material, the second material having a second magnetic susceptibility; and
a marker disposed on the second medical device and formed of a third material, the third material having a third magnetic susceptibility that is different than the first magnetic susceptibility and the second magnetic susceptibility.

2. The medical system of claim 1, wherein the third magnetic susceptibility is greater than the first magnetic susceptibility and the second magnetic susceptibility.

3. The medical system of claim 1, wherein the first material and the second material are the same.

4. The medical system of claim 3, wherein the third material is an alloy that includes less than 1% of iron by weight.

5. The medical system of claim 1, wherein the second medical device can be used coaxially with the first medical device.

6. The medical system of claim 1, wherein the marker comprises a plurality of markers.

7. The medical system of claim 6, wherein the plurality of markers comprises a first subset of markers, a second subset of markers, and a third subset of markers, each marker in the first subset of markers formed of the third material, each marker in the second subset of markers formed of a fourth material, and each marker in the third subset of markers formed of a fifth material, the fourth material having a fourth magnetic susceptibility that is different than the first magnetic susceptibility and the second magnetic susceptibility, the fifth material having a fifth magnetic susceptibility that is different than the first magnetic susceptibility and the second magnetic susceptibility.

8. The medical system of claim 7, wherein each of the first material and the second material is Inconel 625.

9. The medical system of claim 7, wherein each marker in the first subset of markers is formed of a nickel.

10. The medical system of claim 7, wherein each marker in the second subset markers is formed of a first alloy that includes less than 1% of iron by weight.

11. The medical system of claim 7, wherein each marker in the third subset of markers is formed of a second alloy that includes less than 1% of iron by weight.

12. The medical system of claim 7, wherein each of the first material and the second material is titanium;
wherein each marker in the first subset of markers is formed of a stainless steel;
wherein each marker in the second subset markers is formed of a first alloy that includes less than 1% of iron by weight; and
wherein each marker in the third subset of markers is formed of a second alloy that includes less than 1% of iron by weight.

13. The medical system of claim 7, wherein each of the first material and the second material is titanium;
wherein each marker in the first subset of markers is formed of a stainless steel;
wherein each marker in the second subset markers is formed of a first cobalt chromium alloy; and
wherein each marker in the third subset of markers is formed of a second cobalt chromium alloy that is different than the first cobalt chromium alloy.

14. A medical device useful in performing treatment under magnetic resonance imaging comprising:
an elongate member having a proximal end, a distal end, and a main body formed of a first material having a first magnetic susceptibility; and
a plug disposed on the elongate member, the plug comprising a second material disposed within a third material, the second material having a second magnetic susceptibility, the third material having a third magnetic susceptibility, the second magnetic susceptibility being different than the first magnetic susceptibility and the third magnetic susceptibility.

15. The medical device of claim 14, wherein the first material is different than the second material and the third material.

16. The medical device of claim 14, wherein the second material comprises magnetically susceptible particles; and
wherein the third material comprises an epoxy resin.

17. The medical device of claim 16, wherein the magnetically susceptible particles comprise magnetically susceptible metallic particles.

18. The medical device of claim 17, wherein the magnetically susceptible metallic particles comprise a stainless steel alloy.

19. A method of performing treatment under magnetic resonance imaging comprising:
positioning a patient within a magnetic resonance scanner;
scanning a first portion of the patient using the magnetic resonance scanner;
obtaining a magnetic resonance image of the first portion of the patient;
identifying a tissue that has predefined characteristics using the magnetic resonance image;
selecting a procedure to treat the tissue based upon the predefined characteristics;
selecting MR sequences and/or parameters to perform the selected procedure;
selecting a medical device to accomplish performance of the selected procedure;
while the patient is positioned within the magnetic resonance scanner used to scan the first portion of the patient, advancing the medical device into a bodily passage and to the tissue while scanning a second portion of the patient that includes the medical device using the magnetic resonance scanner;
obtaining a magnetic resonance image of the second portion of the patient that includes the medical device;
confirming the position of the medical device within the bodily passage; and
performing treatment.

20. The method of claim 19, wherein the medical device comprises:
an elongate member having a proximal end, a distal end, and a main body formed of a first material having a first magnetic susceptibility; and
a plug disposed on the elongate member, the plug comprising a second material disposed within a third material, the second material having a second magnetic susceptibility, the third material having a third magnetic susceptibility, the second magnetic susceptibility being different than the first magnetic susceptibility and the third magnetic susceptibility.
